(19) United States
(12) Patent Application Publication
(10) Pub. No.: US 2025/0009942 A1
(43) Pub. Date: Jan. 9, 2025

(54) BIODEGRADABLE PIEZOELECTRIC NANOFIBERS

(71) Applicant: University of Connecticut, Farmington, CT (US)

(72) Inventors: Thanh Duc NGUYEN, South Windsor, CT (US); Yang LIU, Willimantic, CT (US)

(21) Appl. No.: 18/888,959

(22) Filed: Sep. 18, 2024

Related U.S. Application Data
(63) Continuation of application No. PCT/US2023/016073, filed on Mar. 23, 2023.
(60) Provisional application No. 63/324,418, filed on Mar. 28, 2022.

(51) Int. Cl. A61L 31/14; A61L 31/12; A61N 7/00; C08G 63/08; C08K 5/17; D01D 5/00; D01F 1/10; D01F 6/62

(52) U.S. Cl. CPC A61L 31/148; A61L 31/129; A61N 7/00; C08G 63/08; C08K 5/175; D01D 5/0038; D01F 1/10; D01F 6/625; A61L 2400/12; A61N 2007/0026; A61N 2007/0043; D10B 2331/041; D10B 2401/061; D10B 2509/00

(57) ABSTRACT

A method of making a biodegradable piezoelectric composite material is provided. Such a method may include mixing a solution comprising biodegradable polymer, glycine crystal, and at least one solvent. The method further comprises electrospinning the solution and receiving the electrospun solution onto a collector drum having a speed of about 100 RPM to about 4,000 RPM. The resulting biodegradable polymer fibers are then substantially aligned with each other. Also provided is a composite material having glycine crystal and a biodegradable polymer.

### CROSS-REFERENCE TO RELATED APPLICATION

This application is a continuation of International Patent Application No. PCT/US2023/016073, filed on Mar. 23, 2023, which takes priority from U.S. Provisional Patent Application No. 63/324,418, filed on Mar. 28, 2022, the entire contents of which are hereby incorporated by reference.

### STATEMENT OF FEDERALLY SPONSORED RESEARCH

This invention was made with government support under grant number 1635103 awarded by the National Science Foundation. The government has certain rights in the invention.

### FIELD OF THE INVENTION

This disclosure relates to biodegradable piezoelectric nanofibers, and in particular relates to piezoelectric polymer and amino acid materials and their use in medical applications.

### BACKGROUND

Piezoelectric materials, a type of “smart” material that can convert mechanical force into electricity and vice versa, are the core of many medical devices including pressure sensors, actuators, and ultrasonic transducers. In many cases, these devices are implanted inside the body for various applications such as monitoring intra-organ pressures, generating ultrasound to facilitate drug-delivery, ablating diseased tissues, and/or stimulating tissue healing etc.

An appealing potential of piezoelectric devices is their ability to be self-powered by harvesting the body's own motion and thus avoiding the use of toxic batteries often required for medical implants. However, conventional piezoelectric ceramics (e.g., lead zirconate titanate (PZT)) or polymers (e.g., polyvinylidene fluoride (PVDF)) are not degradable and even contain toxic elements (e.g., lead in PZT). Thus, devices made from traditional piezoelectric materials may require an undesired removal surgery that can be invasive and can easily damage delicate tissues directly interfaced with the piezoelectric implants.

Recently, there have been reports on the potential for use of safe and biodegradable piezoelectric glycine, the simplest amino acid crystal, which possesses an extremely high piezoelectric constant, comparable to that of piezoelectric ceramics like PZT. Unfortunately, glycine crystals are brittle, difficult-to-handle, and quickly dissolve in aqueous solutions such as body fluid. Existing attempts to leverage the properties of glycine crystals use solvent-casting methods to integrate piezoelectric glycine crystals with soft polymeric matrices to effectively harvest its piezoelectricity. However, this method lacks adequate control over crystal growth direction, resulting in random orientations of crystal domains/dipole moments and leading to an overall low piezoelectric output from the film despite the high piezoelectric constant in each glycine crystal.

Other deficiencies and inferior characteristics of existing solvent cast implementations are discussed throughout the disclosure as well. Therefore, there is a need for a piezoelectric material utilizing glycine crystals in a flexible and easy-to-use form that can fully exploit the excellent piezoelectric effect of this organic material for medical device applications. There is a further need for a new approach to manufacturing such a material, including a method that can create oriented glycine crystals. There is a further need for such materials usable in transducers, as well as the transducers themselves, as well as methods of delivering therapeutics utilizing such materials through a blood-brain barrier in a subject.

### SUMMARY

One embodiment described herein is a composite material comprising: a biodegradable polymer; and a plurality of glycine crystals embedded in the biodegradable polymer. In one aspect, the biodegradable polymer comprises a plurality of biodegradable fibers. In another aspect, the plurality of biodegradable fibers are substantially parallel-aligned with each other as measured by scanning electron microscopy. In another aspect, each biodegradable fiber has a diameter of about 1 μm to about 5 μm. In another aspect, the biodegradable polymer has a number average molecular weight of about 10 kDa to about 200 kDa. In another aspect, the biodegradable polymer comprises one or more of poly (L-lactic acid) (PLLA), poly (D,L-lactide-co-glycolide) (PLGA), polycaprolactone (PCL), polyglycolic acid (PGA), polyhydroxybutyrate, silk, polyvinyl alcohol, chitosan, or combinations thereof. In another aspect, each glycine crystal has an α-form, a β-form, or a γ-form. In another aspect, each glycine crystal has a diameter of about 1 nm to about 5 μm. In another aspect, the plurality of glycine crystals are substantially parallel-aligned with each other as measured by X-ray scattering. In another aspect, the plurality of glycine crystals are uniformly distributed in the biodegradable polymer. In another aspect, the material comprises a weight ratio of about 0.25:1 to about 1:1, or in some cases, up to 2:1, of the plurality of glycine crystals to the biodegradable polymer (glycine:polymer). In another aspect, the glycine crystals that have the β-form substantially remain in the β-form for at least 30 days in vacuum. In another aspect, the composite material has an elastic modulus of about 10 MPa to about 2,000 MPa. In another aspect, the composite material has a piezoelectric output of greater than 600 kPa as measured by generated acoustic pressure.

Another embodiment described herein is a method of making a biodegradable piezoelectric composite material, the method comprising: electrospinning a mixture comprising a biodegradable polymer, a plurality of glycine crystals, and at least one solvent onto a collector drum having a speed of about 100 rpm to about 4,000 rpm to provide a piezoelectric composite material as described herein. In one aspect, electrospinning is performed at a voltage of about 10 kV to about 25 kV. In another aspect, electrospinning is performed at a flow rate of about 2 ml/h, at a humidity of about 30% to about 50%, or a combination thereof.

Another embodiment described herein is an ultrasonic transducer comprising: a first metal electrode; a second metal electrode; the composite material as described herein positioned between the first metal electrode and the second metal electrode; and an encapsulation layer covering the first metal electrode, the second metal electrode, and the composite material. In one aspect, the encapsulation layer comprises a biodegradable polymer. In another aspect, the first metal electrode, the second metal electrode, or both are electrically coupled to a wire. The ultrasonic transducer of clause 20, wherein the first metal electrode and the second metal electrode are electrically coupled to an ultrasonic generator through the wire.

Another embodiment described herein is method of delivering a therapeutic through a blood-brain barrier in a subject in need thereof, the method comprising: applying the ultrasonic transducer described herein to a craniotomy defect of the subject; transmitting an ultrasonic wave signal through the wire; and delivering a pulsed acoustic pressure to the craniotomy defect.

In one aspect, the method further comprises administering to the subject a therapeutic intravenously after transmitting the ultrasonic wave signal. In another aspect, the ultrasonic wave signal is driven at about 1 MHz to about 5 MHz.

Various embodiments and aspects of the inventions described herein are summarized by the following clauses:

Clause 1. A composite material comprising:
a. a biodegradable polymer; and
b. a plurality of glycine crystals embedded in the biodegradable polymer.

Clause 2. The composite material of clause 1, wherein the biodegradable polymer comprises a plurality of biodegradable fibers.

Clause 3. The composite material of clause 2, wherein the plurality of biodegradable fibers are substantially parallel-aligned with each other as measured by scanning electron microscopy.

Clause 4. The composite material of clause 2 or 3, wherein each biodegradable fiber has a diameter of about 1 μm to about 5 μm.

Clause 5. The composite material of any one of clauses 1-4, wherein the biodegradable polymer has a number average molecular weight of about 10 kDa to about 200 kDa.

Clause 6. The composite material of any one of clauses 1-5, wherein the biodegradable polymer comprises one or more of poly (L-lactic acid) (PLLA), poly(D,L-lactide-co-glycolide) (PLGA), polycaprolactone (PCL), polyglycolic acid (PGA), polyhydroxybutyrate, silk, polyvinyl alcohol, chitosan, or combinations thereof.

Clause 7. The composite material of any one of clauses 1-6, wherein each glycine crystal has an α-form, a β-form, or a γ-form.

Clause 8. The composite material of any one of clauses 1-7, wherein each glycine crystal has a diameter of about 1 nm to about 5 μm.

Clause 9. The composite material of any one of clauses 1-8, wherein the plurality of glycine crystals are substantially parallel-aligned with each other as measured by X-ray scattering.

Clause 10. The composite material of any one of clauses 1-9, wherein the plurality of glycine crystals are uniformly distributed in the biodegradable polymer.

Clause 11. The composite material of any one of clauses 1-10, wherein the material comprises a weight ratio of about 0.25:1 to about 1:1 of the plurality of glycine crystals to the biodegradable polymer (glycine:polymer).

Clause 12. The composite material of any one of clauses 7-11, wherein the glycine crystals that have the β-form substantially remain in the β-form for at least 30 days in vacuum.

Clause 13. The composite material of any one of clauses 1-12, wherein the material has an elastic modulus of about 10 MPa to about 2,000 MPa.

Clause 14. The composite material of any one of clauses 1-13, wherein the material has a piezoelectric output of greater than 600 kPa as measured by generated acoustic pressure.

Clause 15. A method of making a biodegradable piezoelectric composite material, the method comprising:
a. electrospinning a mixture comprising a biodegradable polymer, a plurality of glycine crystals, and at least one solvent onto a collector drum having a speed of about 100 rpm to about 4,000 rpm to provide a piezoelectric composite material according to any one of clauses 1-14.

Clause 16. The method of clause 15, wherein electrospinning is performed at a voltage of about 10 kV to about 25 kV.

Clause 17. The method of clause 15 or 16, wherein electrospinning is performed at a flow rate of about 2 ml/h, at a humidity of about 30% to about 50%, or a combination thereof.

Clause 18. An ultrasonic transducer comprising:
a. a first metal electrode;
b. a second metal electrode;
c. the composite material of any one of clauses 1-14 positioned between the first metal electrode and the second metal electrode; and
d. an encapsulation layer covering the first metal electrode, the second metal electrode, and the composite material.

Clause 19. The ultrasonic transducer of clause 18, wherein the encapsulation layer comprises a biodegradable polymer.

Clause 20. The ultrasonic transducer of clause 18 or 19, wherein the first metal electrode, the second metal electrode, or both are electrically coupled to a wire.

Clause 21. The ultrasonic transducer of clause 20, wherein the first metal electrode and the second metal electrode are electrically coupled to an ultrasonic generator through the wire.

Clause 22. A method of delivering a therapeutic through a blood-brain barrier in a subject in need thereof, the method comprising:
a. applying the ultrasonic transducer of clause 20 to a craniotomy defect of the subject;
b. transmitting an ultrasonic wave signal through the wire; and
c. delivering a pulsed acoustic pressure to the craniotomy defect.

Clause 23. The method of clause 22, further comprising administering to the subject a therapeutic intravenously after transmitting the ultrasonic wave signal.

Clause 24. The method of clause 22 or 23, wherein the ultrasonic wave signal is driven at about 1 MHz to about 5 MHz.

Also provided is a method of making a biodegradable piezoelectric composite material. Such a method may include mixing a solution comprising biodegradable polymer, glycine crystal, and at least one solvent. The method further comprises electrospinning the solution and receiving the electrospun solution onto a collector drum having a speed of about 100 RPM to about 4,000 RPM. The resulting biodegradable polymer fibers are then substantially aligned with each other.

In some such embodiments, the method further includes growing or retrieving the glycine crystals for the solution, where the glycine crystals are needle-shaped. The method then includes grinding or otherwise crushing the glycine crystals to create glycine particles and incorporating the glycine particles into the solvent as the glycine crystal component.

In some embodiments, the needle-shaped glycine crystals are β-form crystals. In some embodiments, the glycine crystals are grown using a slow evaporation technique. In some embodiments, the glycine particles are ground using a homogenizer to a size of approximately 500 nm. In some embodiments, the glycine particles are themselves elongate crystals.

In some embodiments, the biodegradable polymer is polycaprolactone (PCL), poly (L-lactic acid) (PLLA), poly(D,L-lactide-co-glycolide) (PLGA), polyglycolic acid (PGA), polyhydroxybutyrate, silk, polyvinyl alcohol, chitosan, or combinations thereof.

Also provided are methods for making an ultrasonic transducer utilizing the piezoelectric composite material. In such an embodiment, the method further includes applying a first electrode to a first side of the biodegradable piezoelectric composite material, applying a second electrode to a second side of the biodegradable piezoelectric composite material opposite the first side, and encapsulating the composite material and the first and second electrodes with a biodegradable encapsulating layer.

In some such embodiments, the method further includes determining an idealized functional lifetime of the biodegradable piezoelectric composite material and selecting a thickness for the encapsulating layer based on the idealized functional lifetime.

In some embodiments of such a transducer, the idealized functional lifetime depends on a use case for the biodegradable piezoelectric composite material.

In some embodiments of a transducer, the encapsulating layer is formed from the biodegradable polymer.

Also provided is a method of therapy leveraging the transducer including implanting the ultrasonic transducer adjacent a craniotomy defect of a subject and transmitting an ultrasonic wave signal through a wire in electrical communication with the first or second electrode. The method may further include administering to the subject a therapeutic intravenously after transmitting the ultrasonic wave signal.

### BRIEF DESCRIPTION OF THE DRAWINGS

FIGS. 1-9 show fabrication and characterization of glycine-PCL integrated nanofibers.

FIG. 1 provides a schematic illustration of glycine-PCL nanofibers. The fibers are comprised of PCL matrix filled with aligned glycine crystals, and properties of the film are highlighted in each text box.

FIGS. 2A-H provides photographs of a glycine-PCL nanofiber mat during various key steps of production in accordance with this disclosure.

First, FIGS. 2A and 2B show β-glycine needle-shaped crystals are grown using, e.g., slow evaporation techniques, at different levels of magnification.

As shown in FIGS. 2C and 2D, the grown crystals are then ground in a homogenizer until the size (which may be ˜500 nm, as shown in FIG. 2D) is suitable for an electrospinning process.

As shown in FIGS. 2E and 2F, the ground glycine particles are heavily stirred in PCL solution (PCL+DCM+DMF) until the solution becomes homogenous.

FIG. 2G illustrates an electrospinning process for use in the method described.

As shown in FIG. 2H, electrospinning the solution onto an aluminum foil at approximately 4,000 rpm then provides a highly uniform mat (which may be, for example, 42 cm×12 cm×80 μm).

FIG. 3A-D shows SEM micrographs of PCL (FIGS. 3A and 3B) and glycine-PCL (FIGS. 3C and 3D) fibers. The fibers have excellent alignment over the films and are connected together via the joints. The presence of glycine crystals inside a single nanofiber is clear in the high-magnification view of FIG. 3D.

FIGS. 4A-C show images of stained glycine particles and stained PCL matrix taken as fluorescent images, with the different stains shown in different textures. Extremely small glycine particles are uniformly distributed over the entire nanofiber film of PCL matrix.

FIG. 5 shows FTIR spectra of PCL and glycine-PCL films. Compared to a bare PCL film, the glycine bands (amino and carboxyl groups) appear distinctly at 3155, 2600, and 1583 cm⁻¹ in the glycine-PCL nanofiber sample.

FIG. 6 shows an XRD spectrum of glycine-PCL film. The positions of peaks confirm the existence of both α- and β-phases.

FIG. 7 shows a stress-strain curve of PCL and glycine-PCL mats. The results exhibit substantially low elastic modulus of glycine-PCL sample (E=32 MPa).

FIGS. 8A-D show a glycine-PCL film under various deformations.

FIG. 9 shows an array of images collected at several stages of accelerated degradation of a glycine-PCL film upon immersion into PBS (pH 7.4) and DMEM solutions at 37° C.

FIGS. 10A-17B show a mechanism for piezoelectricity and piezoelectric performance.

FIG. 10A-C show SEM images textured based on orientation and FIGS. 10D-F show the corresponding orientation histograms for solvent-casting, static electrospinning, and dynamic electrospinning of films respectively. FIGS. 10G-I show X-ray scattering patterns obtained by incidence of the X-ray beam perpendicular to the top surface of the films for solvent-casting, static electrospinning, and dynamic electrospinning respectively. As the crystals inside the film become more oriented, the signal changes from full to partial Debye rings. The darker portions of the Debye rings represent aligned crystals and the lighter portions of the Debye ring represent non-aligned crystals, showing that dynamic electrospinning results in higher alignment of crystals, shown in FIG. 10I.

FIG. 11 shows displacement of solvent-casting, static electrospinning, and dynamic electrospinning films, collected under the same voltage (20 Vpp) and at different frequencies (1-4 Hz). The dynamic electrospun sample (4,000 rpm) exhibits significant displacement (˜12 μm), while the solvent-cast and static electrospun (0 rpm) samples exhibit no measurable displacement.

FIGS. 12A-B show reorientation of molecular dipoles in glycine molecules. The synergetic effect of stretching (drawing) and a high external electrical field may attribute to reorientation of molecular dipoles in glycine crystals. In glycine, nitrogen is partially positively charged (δ+) and oxygen is partially negatively charged (δ−), which creates dipole moments pointing from N to O.

FIG. 13 shows surface potential (V) of glycine particles before and after a poling test.

FIGS. 14A-C show vibration modes of PCL and glycine-PCL films measured by a laser vibrometer. As shown, the glycine-PCL film of FIG. 14B shows substantially higher velocity and more displacement.

FIG. 15 shows an ultrasound transmission test on glycine-PCL films with different glycine concentrations (glycine:PCL w/w, shown as different fill textures). The output voltages were recorded by an oscilloscope and then converted to acoustic pressure based on the calibrated sensitivity of the hydrophone.

FIGS. 16A-B show an ultrasound transmitting test for electrospun glycine-PCL, electrospun PLLA, and recently developed solvent-cast glycine-PVA films. Statistical analysis was evaluated with two-way ANOVA followed by Tukey's post-hoc analysis and P-values are available above the data (n=3).

FIG. 17A-19 combine to show an in vitro biocompatibility assessment of glycine-PCL film.

FIGS. 17A-C show a cell imaging assay performed on mouse adipose-derived stem cells (mADSC) incubated with PCL and glycine-PCL films at 37° C. for 48 hours. Live cells (calcein AM) are stained in a first texture, shown as outlines, and dead cells (BOBO™-3 Iodide) are stained in a second texture, shown as dots.

FIGS. 18A-C show flow cytometry results for mADSCs incubated with PCL and glycine-PCL films. The detailed gating strategies for all flow cytometry measurements are included in FIG. 49.

FIG. 19 shows a statistical analysis of the flow cytometry data was assessed with two-way ANOVA followed by Tukey's post-hoc analysis (n=5).

FIGS. 20-28C show z glycine-PCL ultrasonic transducer for in vivo blood-brain barrier (BBB) disruption and therapeutic delivery.

FIG. 20 shows a fully biodegradable glycine-PCL ultrasonic transducer (approximately 5 mm×16 mm×330 μm).

FIG. 21 shows a schematic illustration of a glycine-PCL ultrasonic transducer. The device includes a piezoelectric glycine-PCL mat, molybdenum (Mo) electrodes and wires, and encapsulating PLA layers.

FIG. 22 shows a model of an ultrasonic transducer for BBB disruption. The combination of microbubbles and ultrasound pulses transiently open tight junctions between endothelial cells in brain and increase the permeability of the blood-brain barrier (BBB).

FIG. 23 shows an experiment flow: on time 0 mice were anesthetized, craniotomy was created, the device was implanted, and suturing was performed. After 10 minutes, microbubbles were injected into the tail vein of the mice. 5 minutes later the transducer was operated. The animal received dextran 5 minutes after the sonication process.

FIGS. 24A-D show key steps of the surgical procedure as described above.

FIG. 25 shows a coronal section of a mouse brain illustrating the implant site and the imaged regions.

FIGS. 26A-F show representative confocal fluorescence images of the superficial and the deep regions show the signal of dextran. The images are immunostained for CD-31 to detect blood vessels. The dashed line indicates the site of the implant.

FIG. 27 shows the control of a functional lifetime of glycine-PCL by altering the thickness of the encapsulating layer.

FIGS. 28A-C show images collected at several stages of accelerated degradation of a glycine-PCL transducer upon immersion into 10×PBS buffer (pH 7.4) at 37° C.

FIGS. 29-35 show glycine-PHB nanofibers for high temperature and long term piezoelectric applications.

FIG. 29 shows a representative image of a glycine-PHB mat after electrospinning. The two-ended arrow represents the fibers direction.

FIG. 30 shows a glycine-PHB mat after vacuum drying for 3 days. The film can then be easily peeled from the aluminum substrate.

FIG. 31 shows a SEM micrograph of glycine-PHB fibers drawn from section 31 of FIG. 30, thereby providing a detailed view. The morphology of the fibers is similar to glycine-PCL fibers.

FIGS. 32A-D show hot stage micrographs of glycine-PHB fibers of FIG. 31 at different temperatures. FIG. 32A shows the hot stage micrograph at 100° C., FIG. 32B shows the same at 120° C., FIG. 32C shows the same at 140° C., and FIG. 32D shows the same at 160° C. The fibers are stable even at 140° C. As highlighted in FIG. 32C, as glycine crystals have a higher melting temperature (230° C.) than the PHB matrix.

FIG. 33 shows specific heat flow obtained from differential scanning calorimetry (DSC) for glycine-PHB fibers. DSC results further certify the stability of the fibers up to 140° C.

FIG. 34 shows displacement of glycine-PHB film at 20 Vpp under the actuation test as described in Example 1. The displacement (˜2.5 μm) is lower than glycine-PCL (˜12 μm). This is most likely because of the stiffer nature of PHB which strongly restricts the glycine crystals.

FIG. 35 shows an ultrasound transmitting test for glycine-PHB and glycine-PCL films. Similar to FIG. 34, glycine-PCL film has significantly better performance than glycine-PHB film. The P value was calculated using unpaired two-sided Student's t test (n=3). The graph shows mean (center line)±s.d. (whiskers).

FIGS. 36A-42 show glycine-PVA nanofibers for high temperature and short-term piezoelectric applications.

FIGS. 36A-C show a portion of a fabrication process of a glycine-PVA film.

FIGS. 37A-F show images collected at several stages of degradation of a glycine-PVA film upon insertion into PBS (pH 7.4), shown in FIGS. 37A-C, and DMEM solutions, shown in FIGS. 37D-F, at room temperature.

FIG. 38 is a photograph of a glycine-PVA mat after electrospinning.

FIG. 39A-B show SEM micrographs of glycine-PVA fibers. Growth of glycine crystals on the surface of the fibers is structurally different than glycine-PCL and glycine-PHB films. FIG. 39A provides a detailed view of section 39A of FIG. 38 and FIG. 39B provides a detailed view of section 39B of FIG. 39A.

FIG. 40 is an XRD pattern of glycine-PVA film. The positions of peaks confirm the existence of γ-phase.

FIG. 41 is a thermogravimetric analysis (TGA) of glycine-PVA film. The melting temperature is around 200° C. which is higher than glycine-PHB film (140° C.).

FIG. 42 shows displacement of glycine-PVA film at 20 Vpp under the actuation test. The displacement (˜1.3 μm) is significantly lower than glycine-PCL (˜12 μm).

FIG. 43A shows FTIR measurements for glycine-PCL films with different glycine concentrations. As the concentration of the glycine increases, the intensity of the glycine bands (N—H) at 3155, 2600, and 1583 cm⁻¹ also increases. FIGS. 43B and 43C illustrate Glycine and PCL molecules respectively.

FIGS. 44A-B show XRD stability results of glycine-PCL film over time, and XRD data for different glycine concentrations.

FIG. 44A shows XRD measurements of a glycine-PCL film over 1 month. The sample was kept in vacuum all the time except for a brief analysis under the X-ray machine.

FIG. 44B shows XRD data of glycine-PCL films with different glycine concentrations. Films with higher glycine concentration, show higher intensity β-phase peaks.

FIG. 45A-B show flexibility of a solvent-cast film vs. an electrospun film.

FIG. 45A shows a solvent-cast glycine-PCL film.

FIG. 45B shows an electrospun glycine-PCL film.

FIGS. 46A-D show a degradation profile of glycine-PCL film.

Representative optical images of a degrading glycine-PCL film on days 0, 2, 4, and 6 in DI water, 1×PBS, 10×PBS, and FBS at 37° C. (physiological temperature). Graphical representation of the % weight loss of the film is presented on the right.

FIGS. 46A-B show such a profile for a 0.25:1 glycine:PCL ratio.

FIG. 46C-D show such a profile for a 1:1 glycine:PCL ratio. It can be seen in the graphs that most of the glycine content dissolves in the first 2 days.

FIGS. 47A-B show surface-wetting characterization using contact-angle measurements.

FIG. 47A shows a PCL film.

FIG. 47B shows a Glycine-PCL film. Glycine crystals slightly enhance the wettability by decreasing the contact angle from 120.8° to 109.8°.

FIG. 48A-C show glycine-PCL films fabricated using three methods. The films are fabricated using the following three methods: solvent-casting, static electrospinning (with a collector drum speed of 0 rpm), and dynamic electrospinning (with a collector drum speed of 4,000 rpm). For solvent-casting, the glycine-PCL precursor solution was made at the same concentration of glycine used for the electrospinning, and casted into a mold before vapor drying process.

FIG. 49 shows flow cytometry gating strategy. Cells were gated based on size and granularity using FSC-A vs SSC-A to eliminate debris and clumped cells. Single cells were sub-gated and subsequently live and dead cells were discriminated by the presence of Calcein (494/517 nm) and Ethidium homodimer-1 (528/617 nm), respectively. Single stained cells were examined before each group. Values inside the plots represent the percentages from the parent gate. FSC-A: forward scatter area, SSC-A: side scatter area, SSC-H:side scatter height.

FIG. 50A-E shows brain sections of no stimulation region (internal control). Representative confocal fluorescence images of the no stimulation region show no signal of dextran (compare with darker sections of 51A-E and 52A-E). The PCL, PLLA, and glycine-PCL transducers are implanted on the other side of the brain, so this region is used as an internal negative control. The images are immunostained for CD-31 to detect blood vessels (See vessels outlined in images captioned “Microvessels” for each of FIGS. 50C-E).

FIGS. 51A-E show brain sections of a superficial stimulation region. Representative confocal fluorescence images of the superficial stimulation region for piezoelectric PLLA and piezoelectric glycine-PCL show the signal of dextran (darker regions) whereas PLC group does not show noticeable dextran signal. The leaked dextran across the BBB is visible for PLLA group, FIG. 51C, but much lower than glycine-PCL group, FIG. 51E which shows a larger and darker dextran region. The images are immunostained for CD-31 to detect blood vessels (see vessels outlined in images captioned “Microvessels” for each of FIGS. 51C-E). The dashed line indicates the site of the implant.

FIG. 52A-E show brain sections of deep stimulation region. Representative confocal fluorescence images of the deep stimulation region show no signal of dextran (darker regions) for PCL and piezoelectric PLLA transducers, whereas a remarkable level of dextran can be seen around the microvessels in the brain of the mouse that received the treatment by glycine-PCL transducer. The images are immunostained for CD-31 to detect blood vessels (see vessels outlined in images captioned “Microvessels” for each of FIGS. 52C-E).

FIG. 53 shows a method for making and using a biodegradable piezoelectric composite material in accordance with this disclosure.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, therapeutic, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or therapeutic described herein to a subject after clinical appearance of such disease, disorder, or its symptoms. “Prophylaxis of” or “preventing” a disease, disorder, or the symptoms thereof involves administering a cell, composition, or therapeutic described herein to a subject prior to onset of the disease, disorder, or the symptoms thereof. “Suppressing” a disease or disorder involves administering a cell, composition, or therapeutic described herein to a subject after induction of the disease or disorder thereof but before its clinical appearance or symptoms thereof have manifest.

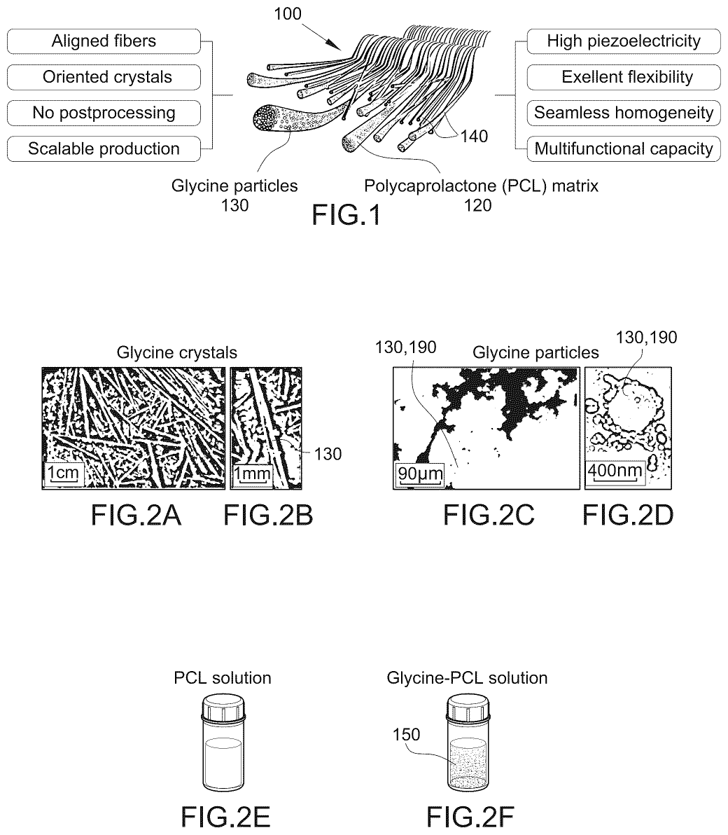

Described herein is a new material as well as a new strategy for material processing and device fabrication to (1) manufacture a biodegradable, flexible, easy-to-use, and highly piezoelectric nanofiber film of glycine crystals embedded inside a biodegradable polymer, such as a polycaprolactone (PCL) polymeric matrix (as shown in FIG. 1), and (2) employ this flexible glycine/polymer nanofiber material to create a powerful ultrasonic transducer which may be implanted into the brain to facilitate drug-delivery through the blood-brain barrier (BBB) for the treatment of brain diseases or disorders. The device can, in some embodiments, then self-degrade after a controllable lifetime to avoid invasive removal brain-surgery.

Accordingly, and as discussed in more detail below, FIG. 1 provides an example of a glycine-PCL integrated nanofiber. A composite material 100 is thereby provided comprising a biodegradable polymer 120, and a plurality of glycine crystals 130 embedded in the biodegradable polymer. While various embodiments are discussed herein and contemplated, Example 2, highlighted below and shown in FIGS. 1-9, relates to such a composite material 100 in which the biodegradable polymer 120 is polycaprolactone, or PCL.

As shown, the composite material 100 typically comprises a plurality of biodegradable fibers 140 formed from the polymer 120. As shown in FIG. 3C, for example, the biodegradable fibers 140 are typically substantially aligned with each other.

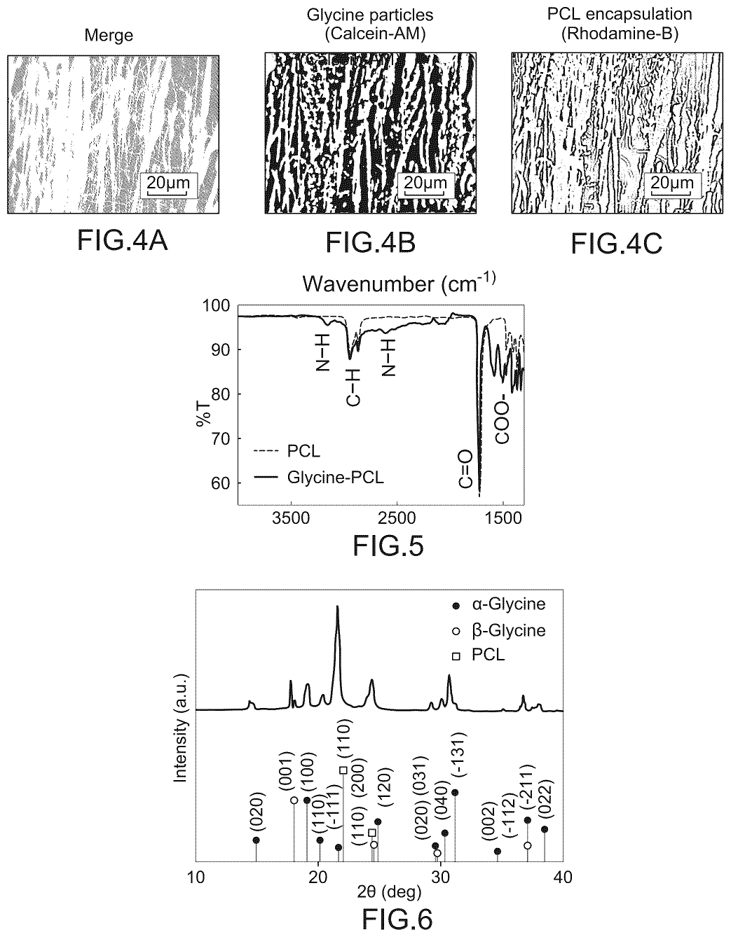

As can be seen in FIG. 3C and in the enlarged view of FIG. 3D, in the embodiment shown, each biodegradable fiber 140 may have a diameter of about 1 μm to about 5 μm. The glycine crystals 130 are typically smaller, but may generate a noticeable bulge 145 when embedded in the polymer 120, as can be seen in FIG. 3D, as well as in FIGS. 4A-C. The glycine crystals 130 may then have diameters between 1 nm and 5 μm.

As shown in those figures, in the embodiment shown, the glycine crystals 130 may be substantially uniformly distributed in the biodegradable polymer 120. As also shown in FIG. 3D, among others, the fibers 140 have excellent alignment and are connected together via joints 155 that support an interconnected film 100.

As discussed below, the glycine crystals 130 referenced may be grown crystals or may be particles 190 of crushed glycine crystals. However, even when crushed, the particles 190 typically have a crystalline form, and are therefore generally referred to as glycine crystals 130 even once crushed.

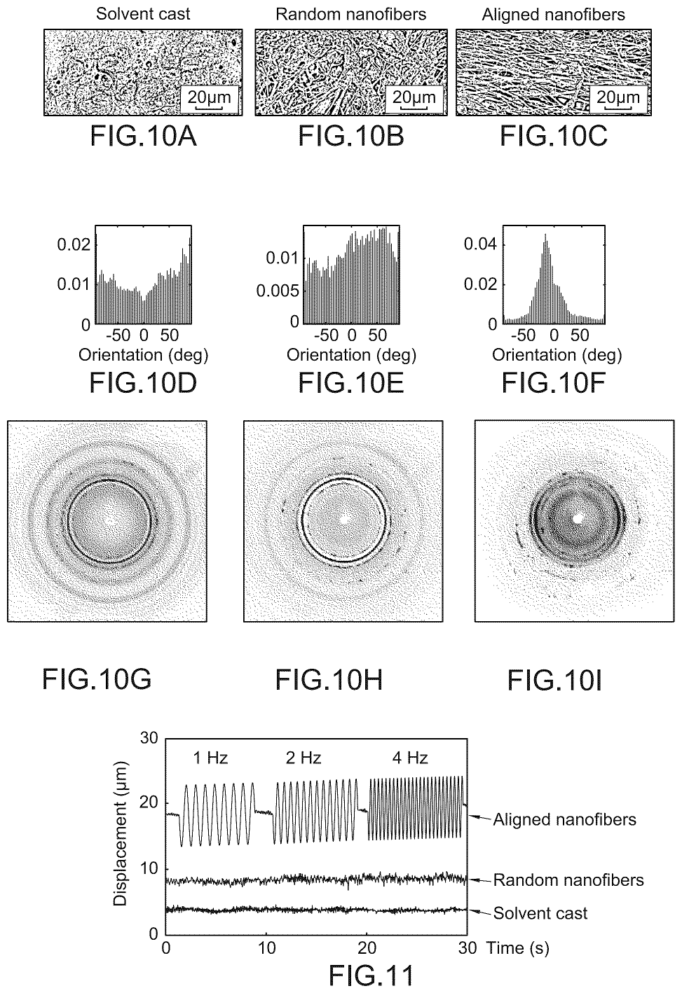

In addition to the alignment of the biodegradable fibers 140, some embodiments provide the glycine crystals 130, or the crystals in particulate form 190, in alignment as well. As shown in FIG. 10I, for example, the methodologies disclosed herein may be used to generate aligned fibers 140 as well as aligned crystals 130 in order to improve piezoelectric characteristics.

While ratios of the biodegradable polymer 120 to the glycine 130 may be varied in different embodiments, in the embodiment shown in FIG. 1, where the biodegradable polymer is PCL, such a ratio is typically about 0.25:1 to 1:1 of the plurality of glycine crystals 130 to the PCL 120. In some embodiments, a higher ratio may be used as well, such as a ratio ranging up to 2:1.

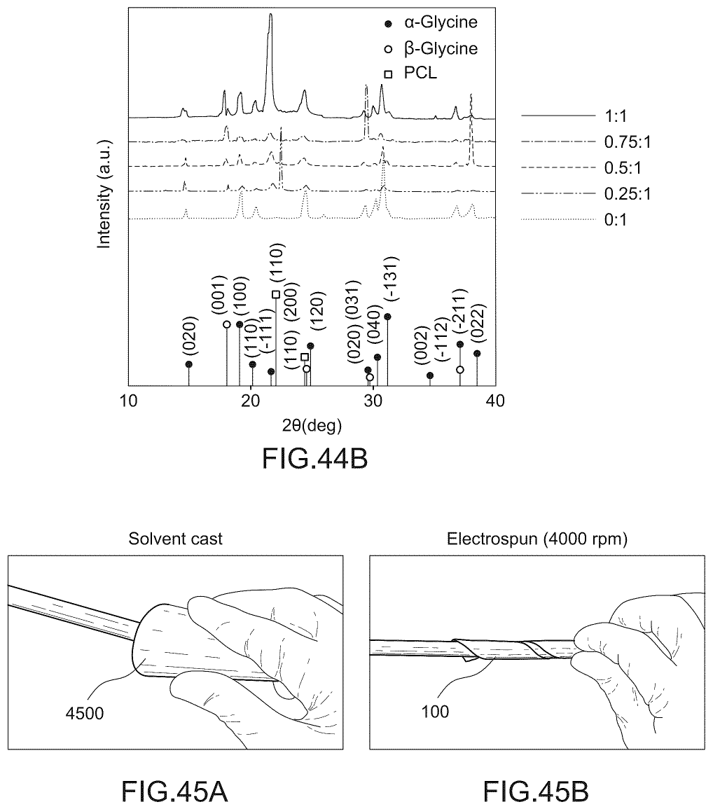

As can be seen in, e.g., FIG. 44A, glycine crystal 130 may be found in the composite material in both α- and β-phases. In the embodiment shown, the PCL nanofibers may stabilize the encapsulated β-glycine which exhibits a strong piezoelectric effect but often is not stable and might otherwise quickly transform into low-piezoelectric α-phase if there is no such encapsulation. In the embodiment shown, glycine crystals having the β form may remain in that form for at least 30 days in a vacuum.

The composite material 100 provided herein may have an elastic modulus of about 10 MPa to about 2,000 MPa.

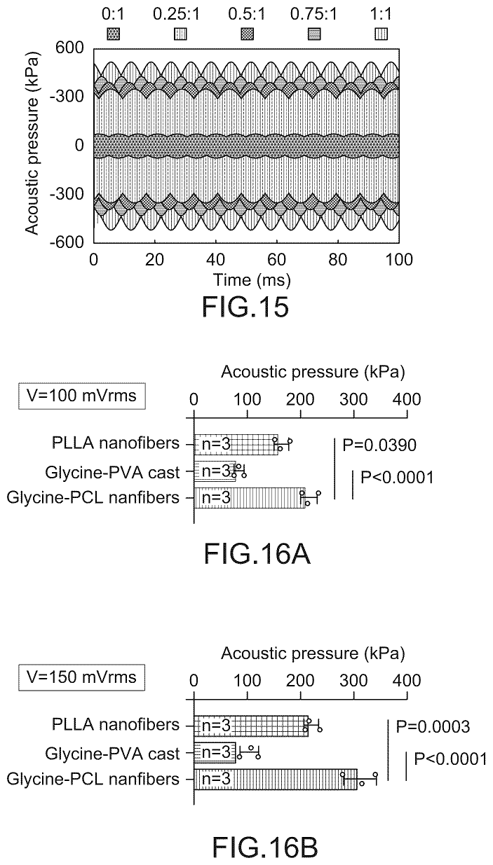

As shown in FIG. 15, for example, the piezoelectric output of the composite material 100 described herein may be measured by generated acoustic pressure. In the embodiment shown, such a material typically has an output greater than 600 kPa as measured by such pressure.

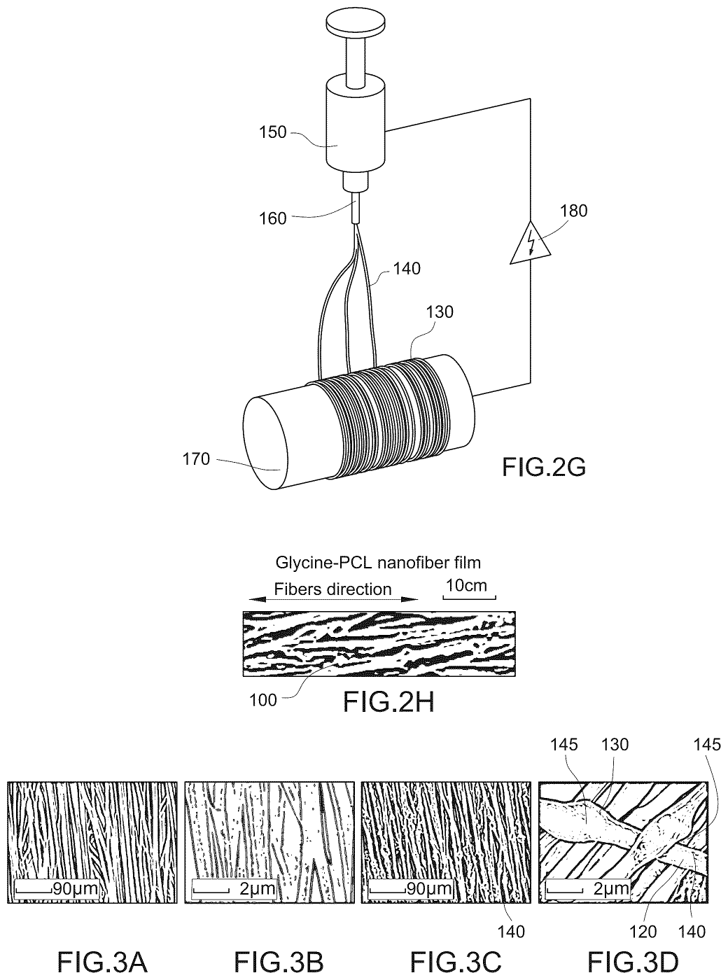

In preparing the biodegradable fibers 140 of the composite material 100 described herein, in some embodiments, electrospinning processes, such as that shown in FIG. 2G, are employed to create nanofibers 140 of glycine-PCL with highly oriented glycine crystals 130, 190 at a large scale. Furthermore, the piezoelectric glycine 130 in this form can produce a high actuation performance (i.e., the ability to convert electricity to movement), superior to that of conventional solvent-cast glycine crystal films. For example, in some embodiments, the flexible piezoelectric glycine-based nanofiber film 100 achieves a high ultrasound performance of 334 kPa, which is approximately three times larger than the acoustic pressure generated by a conventional solvent-cast glycine crystal film under the same applied voltage. Consequently, the nanofibers 140 can generate a substantial acoustic pressure to overcome the BBB and deliver drug model to deeper areas of the brain (˜2 mm) than that achieved by the recently reported biodegradable transducer.

The electrospinning, shown in FIG. 2G, typically involves dispensing a mixture including a solvent 150 from a needle 160 in an electric field generated using a power source 180. In this manner, nanofibers 140 are dispensed onto a collector 170, such as a drum rotating at a speed selected by a user.

Accordingly, as described in more detail below and as shown in FIGS. 2A-2H, in preparing the biodegradable fibers 140, the plurality of fibers are formed by electrospinning a mixture 150 comprising a biodegradable polymer 120, a plurality of glycine crystals 130, 190, and at least one solvent, as shown in FIG. 2F, onto a collector drum 170 having a speed of about 100 RPM to about 4,000 RPM. By providing a drum 170 rotating at such a speed, particularly at the higher speeds closer to 4,000 RPM, the nanofibers 140 are stretched and aligned upon receipt on the drum 170. This results in substantially aligned fibers 140, as discussed elsewhere herein.

During dispensing of the mixture 150 from the needle 160, a voltage is applied by the power source 180 between the needle and collector 170. The voltage may be 14 kV, as noted elsewhere herein, and may typically be between 10 and 25 kV. In addition to the voltage applied by the power source 180 and the rotation speed of the collector drum 170, other variables may be controlled as well. For example, in some embodiments, the electrospinning may be performed at a flow rate of about 2 ml/h from the needle 160. The environment may be maintained at a humidity of about 30% to about 50%.

Especially, it is demonstrated that the glycine-PCL nanofiber device 100 produces a significant level of ultrasound to transiently open the BBB and deliver a drug model (dextran) deep into the brain tissue, exceeding the depth achieved by using a recently reported biodegradable PLLA ultrasonic transducer. By tailoring the encapsulation layer, the functional lifetime of the glycine ultrasound device can be controlled and optimized, which can be useful for different implant applications. Collectively, this highly piezoelectric, flexible, safe, and biodegradable glycine nanofiber film 100 could have a significant impact on medicine by offering many applications in different fields ranging from drug-delivery, tissue stimulation, cellular engineering to medical implant devices such as sensors, actuators, or transducers 2000.

It will be apparent to one of ordinary skill in the relevant art that suitable modifications and adaptations to the materials, methods, processes, and applications described herein can be made without departing from the scope of any embodiments or aspects thereof. The materials and methods provided are exemplary and are not intended to limit the scope of any of the specified embodiments. All of the various embodiments, aspects, and options disclosed herein can be combined in any variations or iterations. The scope of the materials, methods, and processes described herein include all actual or potential combinations of embodiments, aspects, options, examples, and preferences herein described. The ratios of the mass of any component of any of materials disclosed herein to the mass of any other component in the materials or to the total mass of the other components in the material are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1—Materials & Methods

### Fabrication of Glycine-PCL Integrated Nanofibers

Polycaprolactone (PCL, Polysciences MW 50000) solution, shown in FIG. 2E, was prepared by dissolving PCL in mixture of N,N-Dimethylformamide (DMF, Sigma-Aldrich, anhydrous, ≥99.9%) and Dichloromethane (DCM, Sigma-Aldrich, anhydrous, ≥99.8%), with a 20% w/v concentration. Next, β-glycine crystals were grown using similar method as described in Seyedhosseini et al., “Growth and nonlinear optical properties of β-glycine crystals grown on Pt substrates,” Crystal Growth & Design 14:2831-2837 (2014), which is incorporated by reference herein in its entirety, by dissolving glycine powder (Sigma, electrophoresis, ≥99%) in ultrapure distilled water (invitrogen). Solutions were dropped onto clean glass slides using a micropipette. The droplets were left to dry in ambient conditions until crystallization occurred. See FIGS. 2A and 2B. Crystals 130 were carefully removed from the glass slides and grinded in a microtube homogenizer (BeadBug™) with metal beads for 2 minutes. Grinded crystals 190, shown in FIGS. 2C and 2D were added to the PCL solution of FIG. 2E and were heavily stirred overnight, resulting in the solution of FIG. 2F.

Hybrid electrospinning, as shown in FIG. 2G, was done in a 30±10% relative humidity atmosphere under ambient conditions, as shown in FIG. 2G. The solution flow rate was 2 mL/hr through a flat-tipped 21-gauge needle 160 (Jensen Global). The distance from the needle tip to the collector drum was kept at 3¾″ (˜8.5 cm). The applied electric field was set to 14 kV. The polarized solution was then sprayed at a grounded aluminum drum 170, wrapped in aluminum foil 2900, rotating at 4,000 rpm. Later on, fibrous mats 2910 were vacuum dried for 3 days (to ensure the removal of all solvents) before conducting further tests.

### Scanning Electron Microscopy (SEM)

Samples were deposited on a standard SEM stub (Ted Pella) that was covered with carbon tape (Ted Pella) and then sputter-coated with Au/Pd (5 nm thickness) using a sputter coater (CCU-010, Safematic). The samples were then imaged using a Verios 460L SEM at 15 kV and 2500 magnifications. Data collection and analysis were carried out with xT microscope Control software. ImageJ software (NIH) was used for quantitative analysis of alignment and porosity.

### Transmission Electron Microscopy (TEM)

A 3 μl aliquot of the glycine solution was deposited onto carbon-coated copper grids. Samples were adsorbed for 2 min. and rinsed with 100 μL of 0.5% uranyl acetate aqueous solution. Excessive uranyl acetate was blotted with filter paper and grids were air dried. Grids were visualized by using a FEI Tecnai Biotwin TEM (FEI, Eindhoven, Netherlands) operating at 80 KV.

### Fourier Transform Infrared Spectroscopy (FTIR)

FTIR spectra were acquired by Nicolet Magna 560 (Thermo Fisher Scientific) and attenuated total reflectance (ATR) ZnSe. The samples were cut 10 mm×10 mm and casted directly onto the ATR crystal. The measurements were performed and analyzed at several areas. Prior to each experiment, the diamond lens was cleaned using acetone, followed by deionized water. The FTIR spectra were background subtracted and baseline corrected with OMNIC software v.8.3.103.

### X-Ray Diffraction (XRD)

XRD measurements of nanofiber films 100 were carried out at room temperature using a Bruker D2 Phaser instrument equipped with a high-speed linear detector and Cu-Kα radiation (λ=1.54184 Å). Diffractograms were recorded from 10° to 50° (2θ) with a scan speed of 0.2°/min. The patterns were compared with the ICDD powder diffraction file (PDF) database (PDF 00-032-1702 for α-glycine, 00-002-0171 for β-glycine, and 00-006-0230 for γ-glycine) for phase analysis.

### Wide Angle X-Ray Scattering (WAXS)

2D WAXS frames were acquired at room temperature using an Oxford Xcalibur diffractometer equipped with a CCD detector and Cu-Kα radiation (λ=1.5418 Å) at 40 kV and 40 mA. Scattering data were processed by CrysAlisPro software.

### Mechanical Testing

The stress-strain relationships and tensile moduli of the films 100 were measured by Instron 1350 uniaxial tensile tester that was equipped with a 2525 Series Drop-through 100 N load cell and pneumatic grips. All the samples were prepared according to the ASTM D3822 standard. Data collection and analysis were carried out with Bluehill v.3 software.

### Film Degradation

The films 100 were cut 1 cm×1 cm and each piece was placed in a separate well of a 6-well plate and the well was filled with 5 mL of PBS (Gibco, pH 7.4) or DMEM (Gibco). The 6-well plates were placed on a hotplate (Fisherbrand) at 37° C. and imaged every 10 days.

### Measurements Under Actuation System

Actuation Test (I.E., Film's Displacement Under Applied Electrical Field) was Performed By sandwiching the films between Al foil electrodes and half of the film/Al foil squares were encapsulated in polyimide tape (DuPont). The exposed Al foil electrode leads were then reinforced using copper tape (Ted Pella). The fabricated devices were firmly fixed on an Al beam in the actuation system using polyimide tape. Sinusoid waveforms were applied to the actuators from a function generator (BK Precision, 4054B) with varied voltage (20 V peak-to-peak) and frequency (1-4 Hz). The magnitude of the PLLA actuator's displacement was measured at the center of the exposed films using a laser displacement system (Keyence LK-G37). The laser was connected to a controller (Keyence, LKGD500), that sends the displacement data to a computer via USB. The data was then interpreted using LK Navigator software v. 1.6. Any drift in the measured signal was removed using a baseline function in Matlab 2018b.

### Corona Poling and Surface Potential Measurement

Grinded glycine crystals 130, 190 were mounted on a Mo plate and placed between a grounded substrate and a high voltage (20 kV) needle on the top at 5 cm. Ionized air particles around the needle were accelerated and bombard towards the sample surface, causing electric field across the substrate. The surface potentials of glycine crystals 130 were measured by using a non-contact electrostatic voltmeter (ESVM-NC) at a relative humidity of 30%. Later, the samples (n=5 for each group) were positioned on an anti-static mat (Bertech, 1059-2X3BKT) to increase the accuracy of the measurement. The voltmeter was grounded and was held 5 mm from the sample surface.

### Vibrometer Test

A laser vibrometer (Polytec, PSV-I-500), mounted on an optical table and controlled with PSV 9.4 H-Acquisition software, was used for this experiment to measure the out-of-plane velocity of the films. All samples were held flat during the experiment using a pair of custom metal clamps designed to bolt into an optical table. The clamps were secured using metal bolts, which were tightened with a torque wrench (Neiko, 03727A) set to 1 Nm. A chirp waveform was generated by the vibrometer. The amplitude of the voltage waveform from the vibrometer was increased using a voltage amplifier (Piezo.com, EPA-104). The voltage output of the amplifier was then connected to a 50Ω splitter, with one output of the splitter connected to the device with a 50Ω BNC to alligator clip cable. The second output of the splitter connected to an RTM 30004 oscilloscope (Rhode and Schwarz) to ensure the voltage applied to all samples was at 37±1 Vpp. The vibrometer settings were as follows: the vibrometer scanned for vibration frequencies from 100-2000 Hz, applied a chirp waveform with frequencies of 100-2000 Hz, and had a resolution of 20 mm/s and a fast Fourier transform (FFT) size of 800.

### Ultrasound Transmission Test

A capsule hydrophone (HGL-0400, Onda Corp) and the transducers were mounted and immersed in a deionized (DI) water tank. DI water was selected as a testing medium because its acoustic impedance value is close to human tissue. The hydrophone was placed 2 cm away from the transducer and was aligned with the transducer to maximize the harvested signal. A continuous sinusoidal wave at 1 MHz with the amplitude of 0.1 Vrms from the function generator (BK Precision, 4054B) then amplified by an RF power amplifier (E&I RF Amplifier 1040L) was applied to the transducer. The acoustic pressure was recorded by an 8-channel oscilloscope (PicoScope 4000) as an output voltage. These voltage values were converted to acoustic pressure based on the calibrated sensitivity of the hydrophone.

### Cell Imaging Assay

Mouse adipose-derived stem cells (mADSC, iXCells Biotechnologies) were cultured in Dulbecco's Modified Eagle's Medium supplemented (Gibco) with 10% fetal bovine serum (Gibco) and 1% penicillin-streptomycin (Gibco) at 37° C. and 5% CO₂. At 80-90% confluence of passage 4, the cells were detached from the flasks using Trypsin/EDTA (Gibco) and seeded onto the electrospun films at a seeding density of 5×10⁶ cells/mL. After 48 h, the cells were stained with a LIVE/DEAD™ cell imaging kit (Invitrogen). Live/dead images were taken using Leica SP8 confocal laser microscope (Leica Microsystems, Germany) with excitation/emission 488 nm/515 nm and excitation/emission 570 nm/602 nm.

### Flow Cytometry

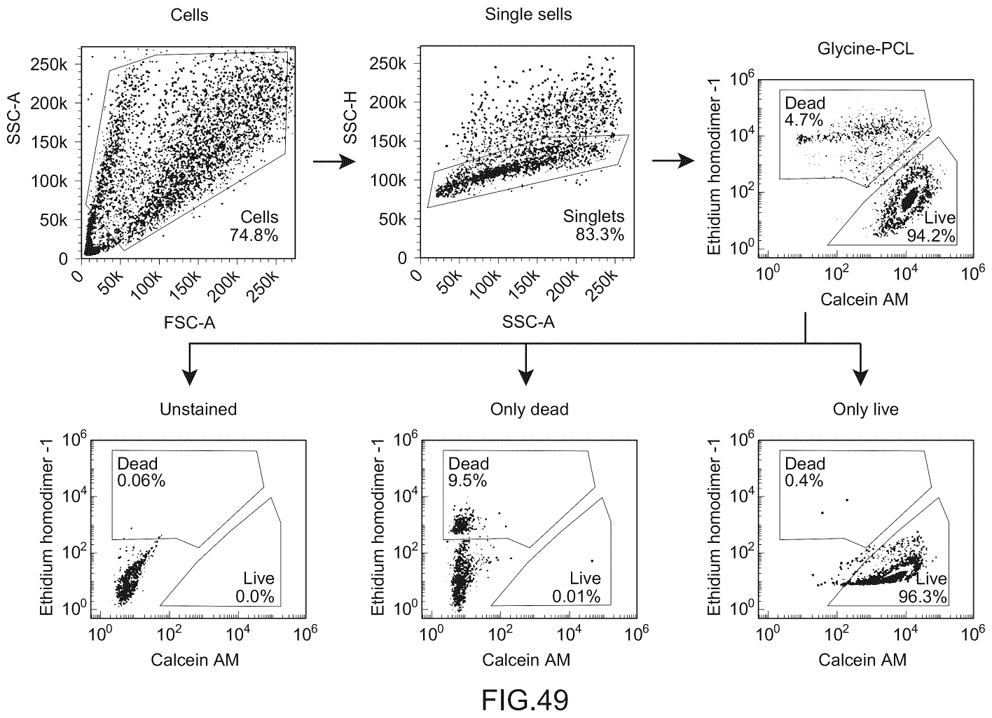

mADSCs were purchased and cultured. The cells were incubated with the electrospun films for 22 h. Next, the medium was removed, and the cells were trypsinized and prepared for flow cytometry analysis on the BD LSRFortessa X-20 cytometer. Flow data were acquired and analyzed with BD FACSDive and FlowJo software. LIVE/DEAD™ viability/cytotoxicity kit (Invitrogen) was used for flow cytometry. The representative flow cytometry gating strategy used in this study is provided in FIG. 49. Statistical analyses were performed with GraphPad Prism (GraphPad Software).

### Fabrication of Biodegradable Ultrasonic Transducers 2000

The electrospun mats 100 were cut into 5 mm×5 mm films. Encapsulating PLA films 2030 were obtained from compression molding using a Carver Press (3850-1011) at 200° C. and quenching using dry ice. Two 5 mm×5 mm squares attached to 1 mm wide wires were cut out of a 25 μm thick sheet of Molybdenum (ESPI Metals) using a pair of scissors. The electrodes 2010, 2020 were then hot embossed into PLA using the Carver Press at 110-130° C. The films 100 were sandwiched between the two electrodes 2010, 2020 that were embossed in PLA, and verification of resistance was done using a multimeter (Extech, MN35). All of the edges of the encapsulator were then sealed three times using a thermal bag sealer (ULINE, H-161) for 5 seconds.

### Surgical Procedure

All animal care and procedures were approved by the University of Connecticut Institutional Animal Care and Use Committee (IACUC). The experiment was conducted on 6 male CD-1 mice (Charles River) which were divided into three groups (n=2). The transducers were sterilized in 70% ethanol for 30 minutes and then exposed to UV light for another 30 minutes. A 5 mm diameter craniotomy defect was created on left side of the midline suture of the mouse anesthetized with ketamine (Covetrus)/xylazine (Covetrus). A drilling tool (Stoelting) was used to cut through the bone while keeping the dura intact. A 5 mm×5 mm single element ultrasound transducer 2000 was implanted on the craniotomy in the mouse's skull. The mouse was allowed to rest for 10 minutes after the defect was sutured. A bolus of 25 μL ultrasound contrast agent (VisualSonics, VS-11694) that contained microbubbles (2× 10⁸ bubbles/mL) was injected into the tail vein of the mice approximately 5 minutes before the sonication. The transducer was operated at 1 MHz, burst rate at 10 Hz with a duty cycle of 20%, generating an acoustic pressure at 0.3 MPa in a series of two shots lasting 30 seconds each with a 30 second break between each shot. The animal received dextran (10 kDa, FITC-Lysine Fixable, Thermo Fisher) 5 minutes after the sonication process. Specifically, 100 μL of dextran was retro-orbitally injected on each eye of the mice (200 μL in total for an animal). After the injection, the mice were allowed to rest for an hour before being transcardially perfused.

### Brain Tissue Processing

Mice were anesthetized with an intraperitoneal injection of ketamine/xylazine. Following exposure of the heart by left anterolateral thoracotomy, the mouse was transcardially perfused (via the left ventricle) first with 20 mL of saline, to flush out the blood, and then with 10 mL of 4% paraformaldehyde (PFA). Each brain was removed carefully from the skull, and post-fixed in 4% PFA overnight at 4° C. The samples were cryoprotected in 15% sucrose and then in 30% sucrose solution until it sunk to the bottom of the container prior to freeze-embedding. The brains were placed in O.C.T. (optimal cutting temperature, Fisher HealthCare) compound and rapidly frozen to −80° C. Subsequently, 30 μm coronal cryosections were obtained using a Leica cryostat (maintained at −25° C.) on precleaned microscope slides (Epredia).

### Immunohistochemistry and Confocal Microscopy

Sections were permeabilized with 0.5% Triton X-100 in PBS for 40 min, and nonspecific binding blocked by incubation with 1× Powerblock® (BioGenex) in UltraPure™ distilled water (Gibco) for 10 minutes. To identify blood vessels, the microvascular endothelium was stained using rat anti mouse CD31 (BD Pharmingen; 1:150 dilution), followed by incubation with goat anti-rat Alexa® 555 (Life Technologies; 1:300) as secondary antibody. The sections were washed in PBS prior to mounting in Mowiol®. Confocal images were acquired using a Leica SP8 confocal microscope.

### Functional Lifetime and Degradation of Glycine-PCL Transducer

The acoustic pressure generated by the devices was recorded by the hydrophone in the transmitting experiment. The device was encapsulated in 90 μm, 160 μm, and 260 μm thick PLA layers. They were immersed in PBS at 37° C. for degradation testing. The devices were removed, then, rinsed with deionized water and the acoustic pressure, under the input condition used for its initial calibration, was measured every 24 hours. This process was repeated with each device until failure. Subsequently, the samples were left in PBS for degradation analyses.

### Example 2—Characterization of Glycine-PCL Integrated Nanofibers

Glycine-PCL integrated nanofibers 140 include a PCL matrix 120 filled with aligned glycine crystals 130 (FIG. 1). Thus, they can take advantage of both high piezoelectricity of glycine crystals 130, and lightweight, flexible properties of the PCL matrix 120 to produce large strain with high piezoelectric output. The fabrication process of glycine-PCL integrated nanofibers 140 and the resulting films 100 is illustrated in FIGS. 2A-H.

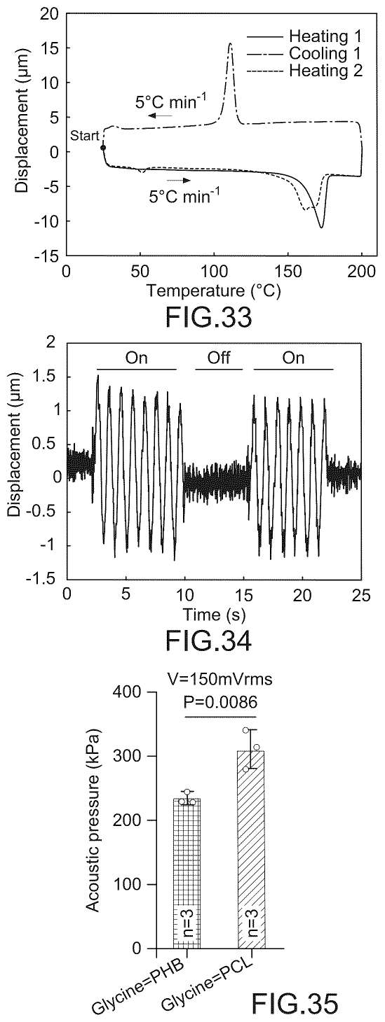

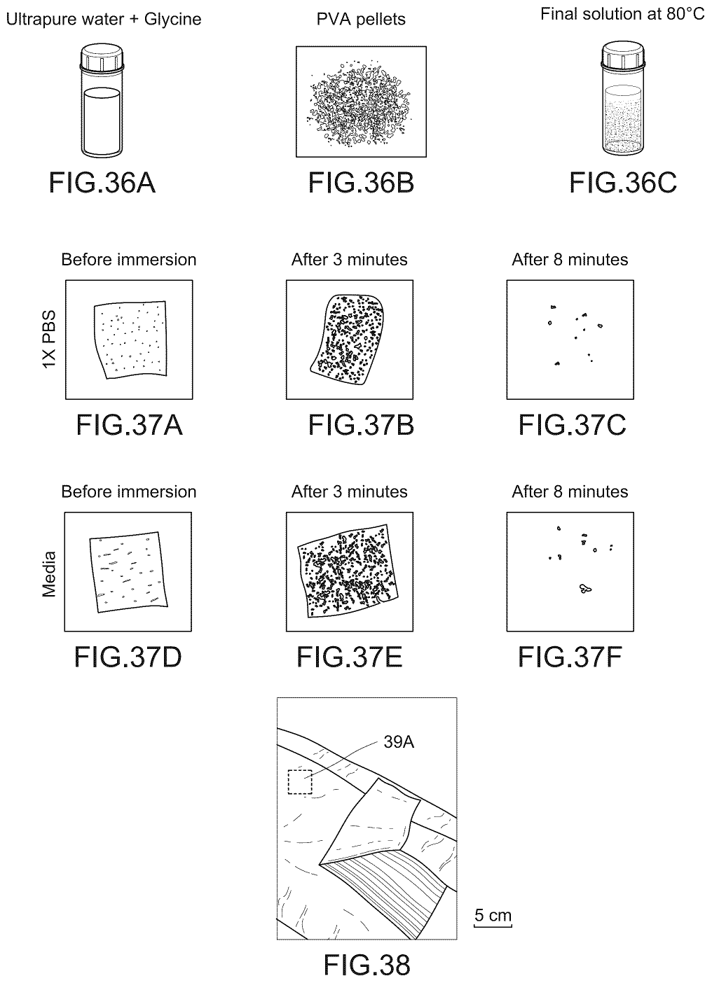

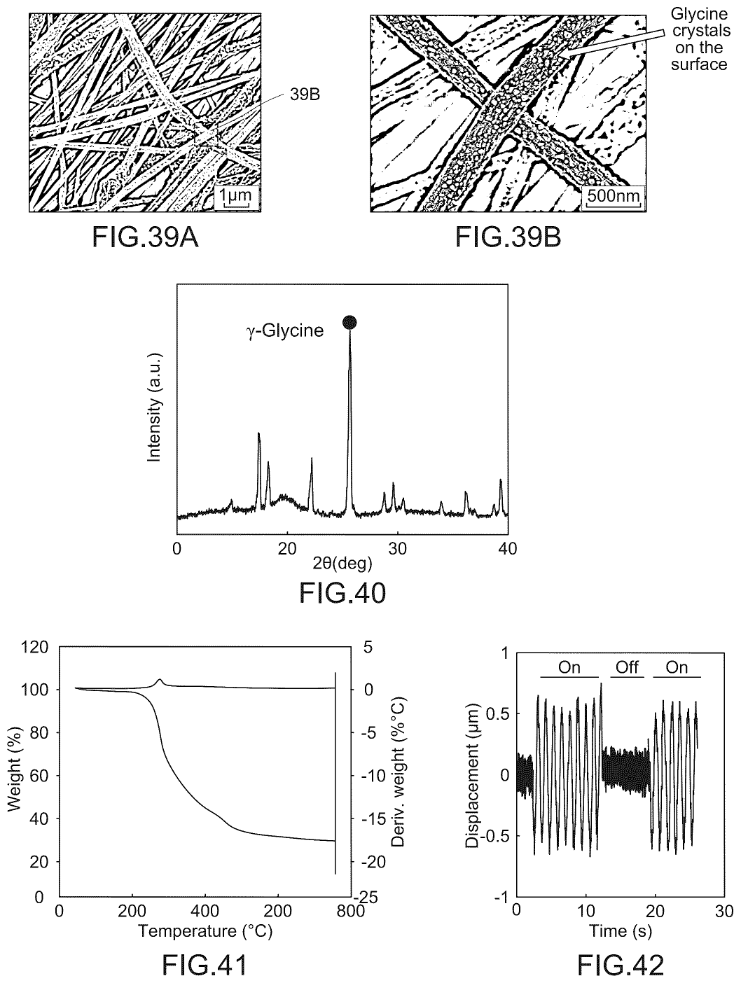

Since β-glycine is the most piezoelectric phase of glycine, β-glycine needle-shaped crystals 130 were grown using slow evaporation technique, as shown in FIGS. 2A-B. The grown crystals 130 were then grinded properly in a homogenizer until particles of the crystal 130, 190 were of a size (which may be ˜500 nm) suitable for the electrospinning process shown in FIG. 2G (TEM image in FIG. 2D). Because the glycine crystals 130 cannot be electrospun directly, a polymeric matrix 120 is necessary for the process. In the embodiment shown, PCL was first chosen because this biodegradable polymer has a useful safety profile and low elastic modulus, which can provide a flexible matrix to carry the highly piezoelectric glycine crystals. PCL has a low melting temperature (Tₘ=57° C.) and thus, for specific applications which require high temperatures, polyhydroxybutyrate (PHB, Tₘ=160° C.) or polyvinyl alcohol (PVA, Tₘ=210° C.) and other polymers could be alternative choices (as shown in FIGS. 29-42).

Accordingly, while the embodiments shown and described are generally presented in terms of PCL as the biodegradable polymer used 120, a wide variety of such polymers are contemplated, including poly (L-lactic acid) (PLLA), poly(D,L-lactide-co-glycolide) (PLGA), polyglycolic acid (PGA), polyhydroxybutyrate, silk, polyvinyl alcohol, chitosan, or combinations thereof.

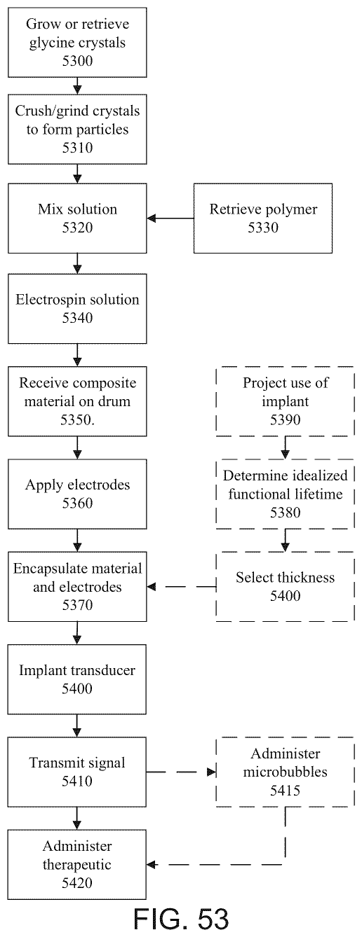

A method for making and using the biodegradable piezoelectric composite material 100 is outlined in FIG. 53 and is illustrated in FIGS. 2A-2H. Accordingly, as shown, the method of making the biodegradable piezoelectric composite material 100 may initially include growing or retrieving glycine crystals 130 (5300) as shown in FIGS. 2A and 2B. The glycine crystals 130 may be needle-shaped, as shown in the figures. Such glycine crystals 130 may be formed using a slow evaporation technique. Once the glycine crystals 130 are acquired, they may be ground or otherwise crushed to create glycine particles 190, as shown in FIGS. 2C and 2D (5310). Such grinding may be done using a homogenizer, and the grinding may be to a target particle side of approximately 500 nm.

The method may then proceed with mixing a solution (5320) that includes a biodegradable polymer 120, separately retrieved (5330), the glycine crystal 130, either in crystal or particle 190 form, and at least one solvent. In some embodiments, it is noted that the glycine particles 190 themselves are elongate crystals 130. As discussed elsewhere herein, the orientation of such elongate crystals 130 may be aligned in the resulting fibers 140. A PCL solution 200 is shown in FIG. 2E and the complete mixture 150 is shown in FIG. 2F. Additional details related to ingredients appropriate for the mixture 150 shown are discussed elsewhere herein.

As shown in FIG. 2G, the solution 150 is then electrospun (5340) and is received (5350) on a collector drum 170 having a speed of about 100 RPM to about 4,000 RPM. Accordingly, the resulting biodegradable polymer fibers 140 are substantially aligned with each other.

The glycine crystals 130 may be β-form crystals.

FIGS. 3A-3D show scanning electron microscopy (SEM) images of PCL and glycine-PCL nanofibers 140 which display the surface morphology of the fibers and the presence of glycine crystals 130 inside a single nanofiber 120, fabricated from an electrospinning process (voltage of 14 kV and drum speed of 4,000 rpm, as shown in FIG. 2G). The electrospinning setup utilized in the embodiment shown enables integrated films 100 with width of ˜12 cm, length of ˜ 42 cm and thicknesses of ˜ 60-100 μm, depending on the running time. The average fiber 120 diameter is ˜ 2 μm, with an excellent alignment over the film 100. The films 100 have internal density of about 9.6×10⁷ fibers per mm², and an overall porosity of ˜20%. It is understood that variations with different characteristics are contemplated as well.

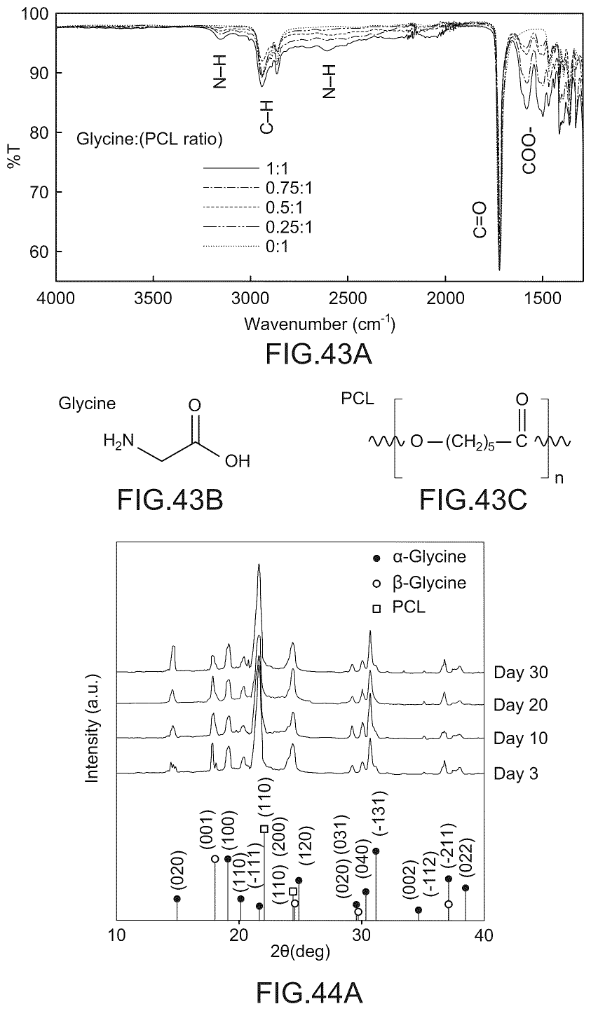

Images of stained glycine particles 190 and a stained PCL matrix 120 provide an insight into the distribution profile of glycine. As seen in FIGS. 4A-4C, with different stain colors shown in different textures, small glycine particles are uniformly distributed over the entire nanofiber film of PCL matrix. FIG. 5 presents Fourier transform infrared (FTIR) spectra of glycine-PCL nanofiber film. Compared to a bare PCL nanofiber film used as a control, the glycine bands (amino and carboxyl groups) appear distinctly at 3155, 2600, and 1583 cm⁻¹ in the glycine-PCL nanofiber sample. Furthermore, the spectra show a strong increase in the intensity of these bands as the glycine concentration increases (as shown in FIG. 43).

To determine the crystal phases, X-ray diffraction (XRD) was performed on glycine-PCL nanofibers 120 (as shown in FIG. 6). The presence of each phase was identified by comparing the corresponding XRD peaks with the ICDD database. The positions of the diffraction peaks confirmed the existence of both α- and β-phases. Notably, the peaks were stable for up to one month in vacuum (as shown in FIG. 44A). This result indicates the benefit of using PCL nanofibers 120 to stabilize the encapsulated β-glycine 130 which exhibits a strong piezoelectric effect but often is not stable and quickly transformed into low-piezoelectric α-phase if there is no such encapsulation. Further, increasing the glycine concentration intensifies the presence of β-peaks (as shown in FIG. 44B).

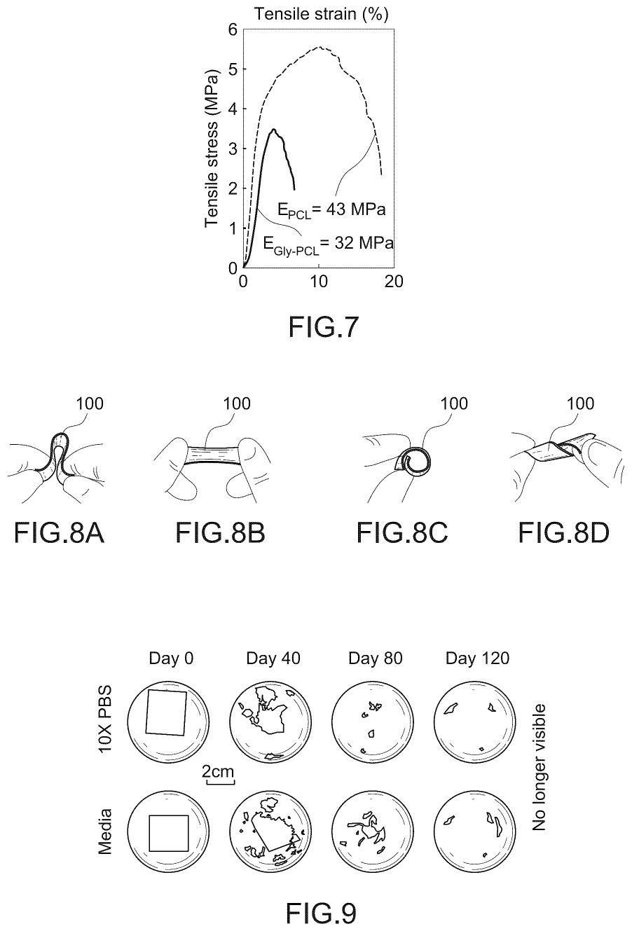

As described, glycine crystals 130 and solvent-casting polymeric film of these crystals are less flexible, rendering them incompatible for practical use in flexible, soft, and implanted devices. A comparison of the flexibility of films manufactured utilizing both techniques is shown in FIGS. 45A-B, with FIG. 45A showing a solvent-cast film 4500 and 45B showing an electrospun film 100. The crystals 130 embedded inside the nanofibrous structure of extremely soft PCL matrix 120 have a considerably improved flexibility and even stretchability. As shown by the stress-strain curves in FIG. 7, glycine-PCL nanofiber mats 100 exhibit a substantially low elastic modulus (E=32 MPa), whereas recently reported solvent-cast glycine films 4500 have an elastic modulus in the range of 3-9 GPa, which indicates a much more rigid structure than the PCL-glycine nanofiber mesh. Photographs of a glycine-PCL nanofiber film 100 intentionally bended, stretched, folded, and twisted many times to highlight the flexibility and mechanical robustness of glycine-PCL film are presented in FIGS. 8A-8D, with FIG. 8A showing bending, FIG. 8B showing stretching, FIG. 8C showing folding, and FIG. 8D showing twisting.

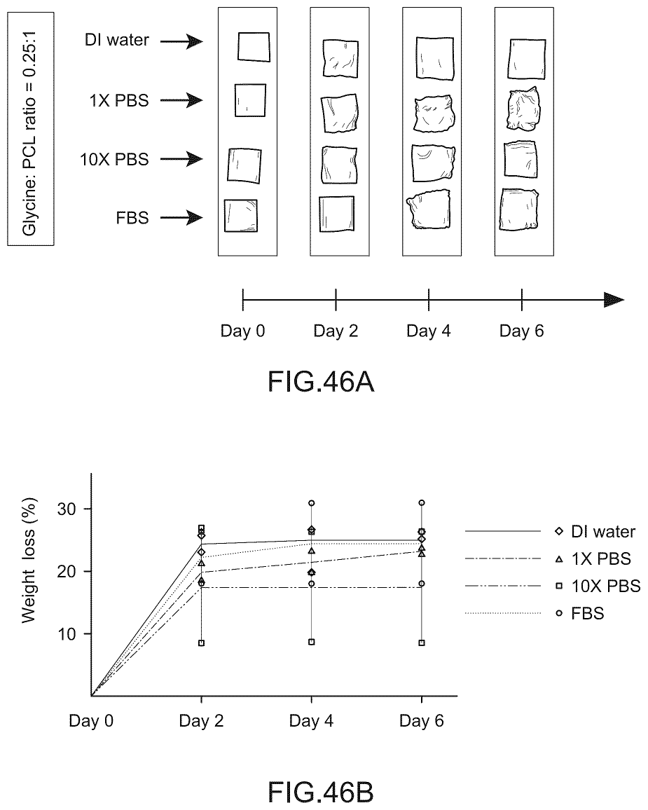

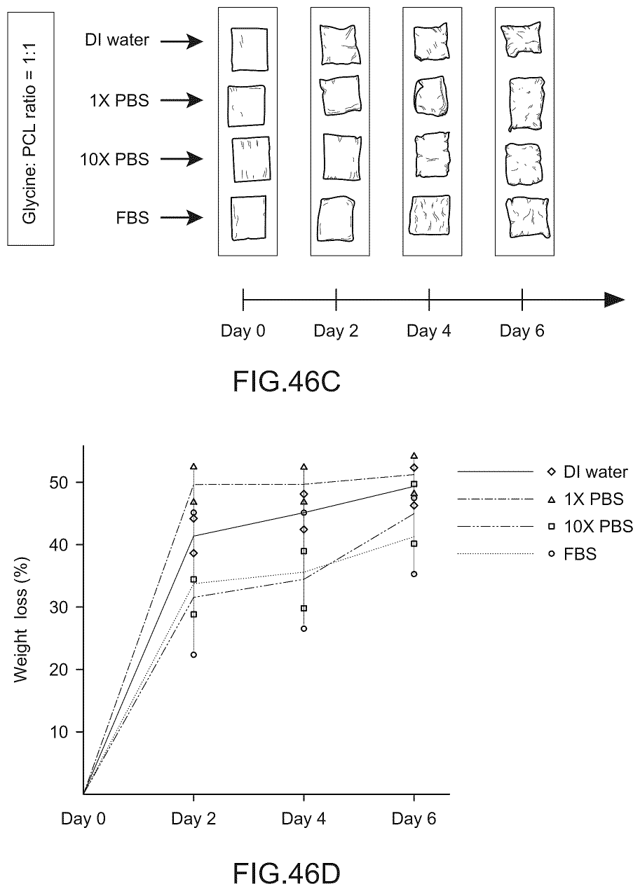

Another advantage, in some scenarios, of the glycine-PCL nanofiber film 100 described herein is its ability to degrade completely when immersed in aqueous solutions, including buffers like phosphate buffered saline (PBS), and biofluids such as Dulbecco's modified eagle medium (DMEM). To illustrate various degradation stages of the nanofiber film, FIG. 9 shows an array of images of glycine-PCL film inserted into wells filled with PBS (pH 7.4) and DMEM and kept at the physiological temperature (37° C.). The integrated nanofiber film 100 degrades uniformly in PBS without fracture. By comparison, the film degrades less uniformly in DMEM, with a tendency to form fragments. Graphical representation of the % weight loss of glycine-PCL film in different solutions is provided in FIGS. 46A-D. Note that for device applications, the glycine film 100 will be encapsulated inside a biodegradable polymer layer 2030 which can be tuned to provide different degradation rates and thus obtain desired functional lifetimes for specific implant applications, as discussed in more detail below.

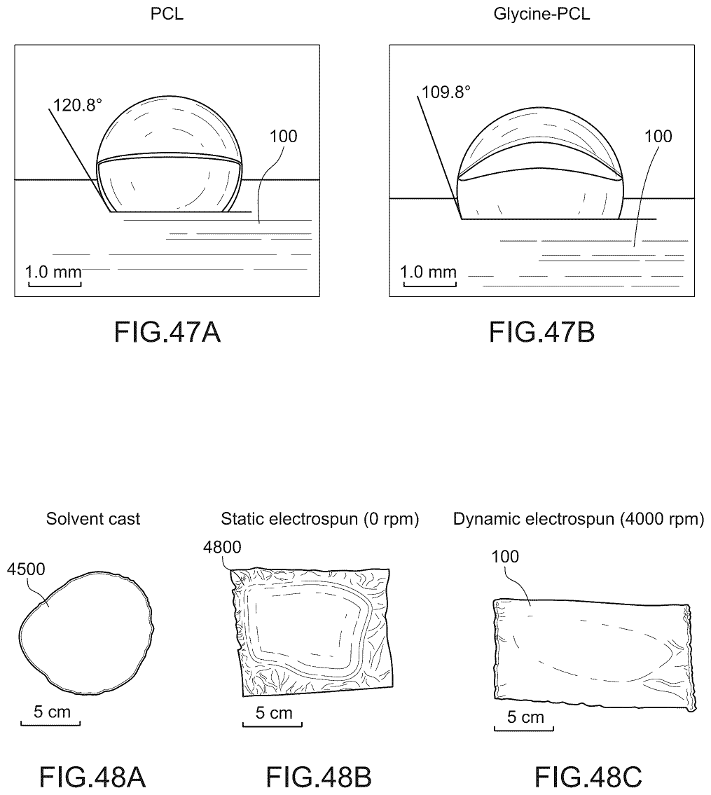

In addition to the degradation study, contact angle measurements were used to characterize the surface wetting properties of PCL 120 and glycine-PCL films 100. It was found that glycine crystals 130 do not considerably modify the wetting properties of the PCL matrix 120, and the glycine-PCL film 100 is slightly hydrophobic, as shown in FIGS. 47A-B.

### Example 3—Potential Origin of Piezoelectricity in Glycine-PCL Nanofibers

Piezoelectricity in organic materials stems from alignment of molecular dipoles. This can be achieved through stretching (drawing) or application of a high external electrical field. Besides, piezoelectric property can be modulated by maximizing the macroscopic alignment of nanofibers over the entire electrospun mat 100. Following these strategies, hybrid electrospinning of glycine particles 130, 190 mixed with PCL solution at high voltage and high speed of the collector 170 in the electrospinning system, shown in FIG. 2G, offers an approach to both stretch and align nanofibers 140, consequently delivering a highly piezoelectric glycine-PCL nanofiber film 100.

To gain insights into how alignment, both at the fiber 140 and crystal 130 levels and under applied electrical field 180, can enhance piezoelectric output, glycine-PCL films 100 were fabricated using the following three methods: solvent-casting 4500, shown in FIG. 48A, static electrospinning 4800 (with a collector drum speed of 0 rpm), shown in FIG. 48B, and dynamic electrospinning 100 (with a collector drum speed of 4,000 rpm), shown in FIG. 48C. For the first method, the glycine-PCL precursor solution 150 was made at the same concentration of glycine 130 used for the electrospinning, and casted into a mold before vapor drying process, as shown in FIG. 48A. A qualitative analysis was performed on orientation of the aforementioned nanofiber films 4500, 4800, 100. As depicted in the orientation shaded SEM images and the corresponding histograms (FIGS. 10A-F), it is clear that the dynamic electrospun film 100 has an exceptional degree of fiber alignment (good for piezoelectricity), compared to the samples of other methods.

Insight into the crystal alignment was obtained through a wide angle X-ray scattering (WAXS) analysis. As the crystals inside the film become more oriented, the WAXS pattern changes from full Debye rings indicating random crystal distribution to partial rings evidencing crystal alignment (FIGS. 10G-I). The less deviation the signal band has from the stretched axis, the more oriented the film is. Thus, electrospinning with a faster collector speed improves the orientation of the crystal domains (also good for piezoelectricity) in each nanofiber.

To evaluate the piezoelectric effect in the glycine-PCL nanofiber mat 100 for actuation performance (which is relevant to the ultrasound application presented later), an alternative electric field (1-4 Hz) was applied to the film 100 sandwiched between two metal electrodes 2010, 2020, which may be aluminum in a test scenario, while the displacement was measured by a laser displacement sensor (i.e., actuation test). The dynamic electrospun sample (4,000 rpm) exhibits an outstanding displacement of ˜12 μm, while the solvent-cast and static electrospun (0 rpm) samples with the same thickness exhibit no measurable displacement (FIG. 11). Results presented herein indicate that the aligned glycine-PCL nanofiber films 100 enable the creation of a powerful actuator which can be used for ultrasonic application at a higher frequency of applied voltage.

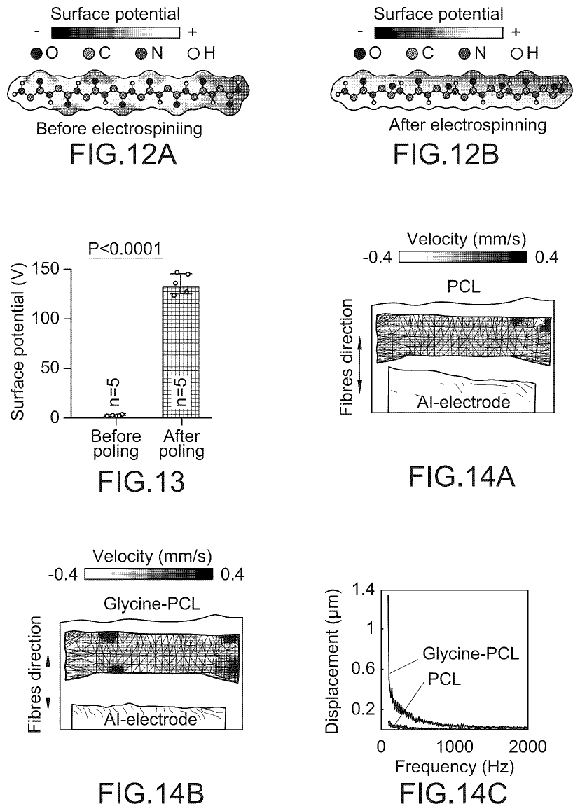

The strong piezoelectric response displayed by glycine-PCL nanofibers 140 in FIG. 11, may rise from reorientation of molecular dipoles after the electrospinning process of FIG. 2G. In glycine molecules, nitrogen is partially positively charged (δ+) and oxygen is partially negatively charged (δ−), which creates dipole moments pointing from N to O. As illustrated in FIGS. 12A-B, it was hypothesized that these dipoles in glycine crystals 130 could be added up from the re-orientation of the oxygen under the high voltage of electrospinning process, resulting in a net dipole moment and a strong piezoelectric response. A similar effect has been reported for peptide and protein-based nanofibers. To somewhat test this hypothesis, a poling test was carried out on glycine particles 130, 190 using corona discharge. The particles 130, 190 were placed on a ground plate and a high voltage (20 kV) was applied between the plate and the top positive nozzle. Surface potentials of glycine particles were measured using non-contact voltmeter before and after poling. As seen in FIG. 13, the charge accumulation rate and surface charge density after the poling were considerably higher than those before poling. Thus, similar to this poling method, during electrospinning, positive ions likely migrate to the negatively charged rotating drum 170 to re-orient dipoles in the glycine molecules 130, 190, and after the film solidification (due to the solvent evaporation), the reoriented dipoles are sustained inside the fibers 140.

### Example 4—Piezoelectric Performance of Glycine-PCL Film

To certify that the piezoelectricity of glycine-PCL nanofiber 140 arises from the glycine crystals 130 and not the PCL matrix 120, the piezoelectric effect of PCL and glycine-PCL fibers 140 were characterized using a laser scanning vibrometer to identify the vibration of the films 100 under applied voltages. Indeed, the glycine-PCL film 100 was clearly demonstrated, whereas bare PCL film 120 does not show any movement (FIG. 14A-C). Following these results and the fact that PCL 120 is not a piezoelectric polymer, PCL film was chosen as a negative control.

In addition to actuation test, it was demonstrated that the same glycine-PCL film 100 can be used to generate ultrasound waves. To do so, an ultrasonic transducer was fabricated by sandwiching the glycine-PCL nanofiber film 100 between two aluminum (Al) electrodes 2010, 2020 and then encapsulating it with a polyimide tape. During the ultrasound transmission testing, a capsule hydrophone was used to measure the acoustic pressure. Glycine-PCL device was driven by a function generator to produce a continuous ultrasonic wave at 1 MHz. As presented in FIG. 15, all glycine-PCL films (with different concentrations of glycine in PCL) generate distinct acoustic pressures while the PCL film without glycine 1500 (the negative control nonpiezoelectric sample) only produces noise. In all these experiments, the highly aligned 1:1 (glycine: PCL w/w ratio) sample 1510 provided the highest conversion signals with the highest output under the same applied voltage, and therefore was used in the remaining examples unless otherwise is stated.

In order to compare the piezoelectric performance of glycine-PCL nanofibers 140 with state-of-the-art biodegradable piezoelectric materials, an ultrasound transmitting test was employed for electrospun PLLA and recently developed solvent-cast glycine-PVA films. The two way ANOVA results clearly demonstrate the superior performance of glycine-PCL nanofiber film compared to the others for actuation applications such as ultrasonic transducers (FIGS. 16A-B).

### Example 5—In Vitro Biocompatibility Assessment of Glycine-PCL Film

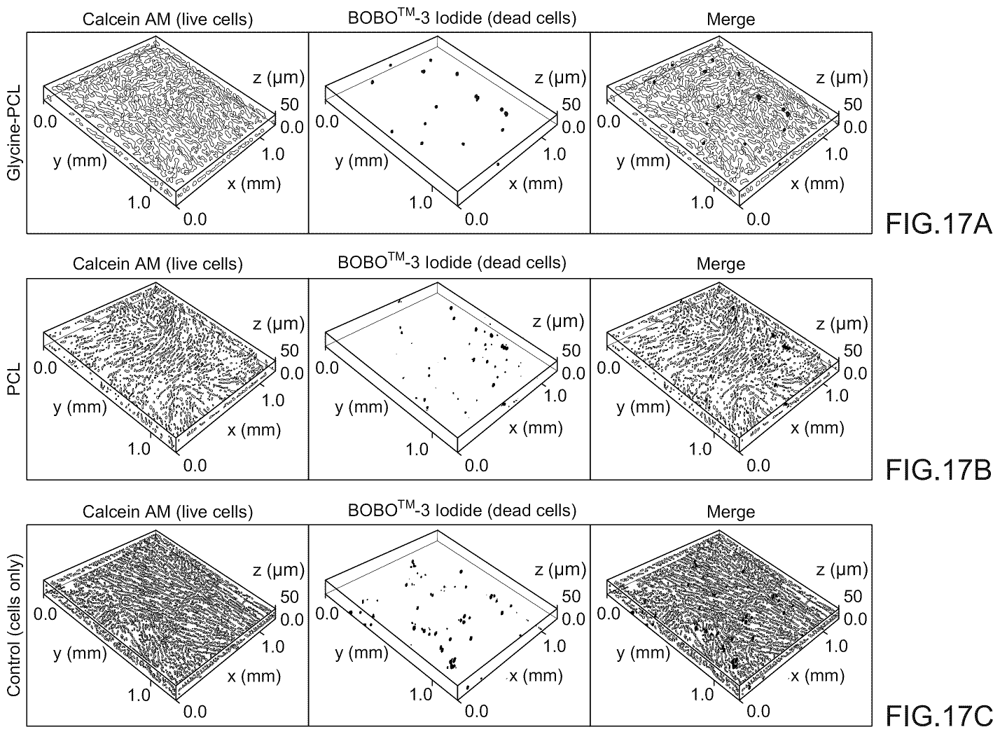

The biocompatibility of glycine-PCL film 100 was examined through two different tests. First, cell imaging assay was used to visualize the effect of the film on the viability of mouse adipose-derived stem cells (mADSC). The mADSCs were seeded on the films at 37° C. for 48 h. Live cells (green stain, calcein AM, shown as outlines in the drawings) are distinguished by the presence of intracellular esterase activity, while dead cells (red stain, BOBO™-3 Iodide, shown as dark dots in the drawings) are detected by the presence of nucleic acids in damaged membranes. As shown in FIG. 17A-C, the number of live/dead cells in glycine-PCL films 100, shown in FIG. 17C, and bare PCL films, shown in FIG. 17B, were similar to that of the control (cells only, shown in FIG. 17A), which demonstrates that glycine-PCL film 100 is not cytotoxic.

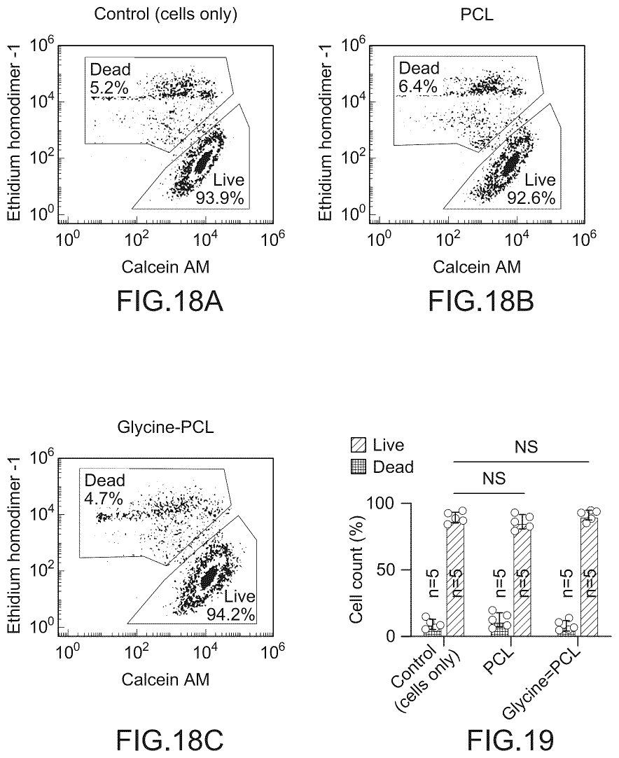

Second, to quantify the number of viable cells in a large number of cells, and further certify the safety of glycine-PCL mat 100, flow cytometry assay was performed with the same mADSCs. All groups (n=5) exhibited a normal behavior, evidencing the non-cytotoxic nature of glycine-PCL film (FIGS. 18A-C and 19). The detailed gating strategies for all flow cytometry measurements are included in FIG. 49.

### Example 6—In Vivo Blood-Brain Barrier Disruption for Drug Delivery Using Glycine-PCL Ultrasonic Transducer

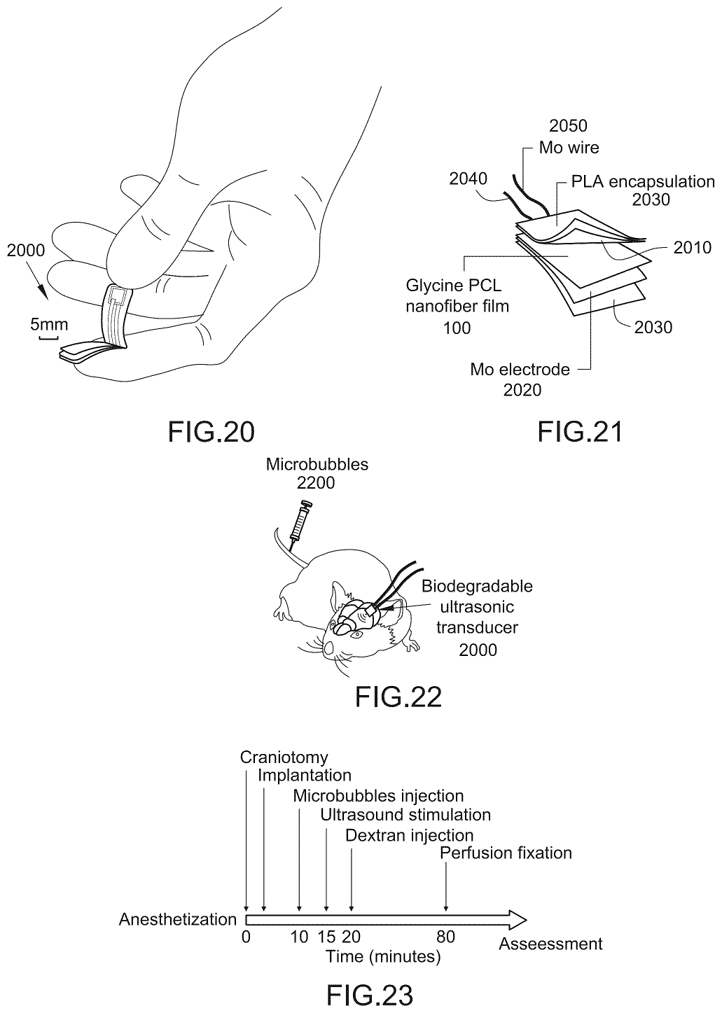

As an example of the usage of the glycine-PCL piezoelectric film in an implantable device, FIG. 20 presents a fully biodegradable ultrasonic transducer 2000 (5 mm×16 mm×330 μm), made from the glycine-PCL film 100. Implanted ultrasound transducers have recently emerged as an effective and safe method to facilitate the repeated blood-brain barrier (BBB) opening for the delivery of drugs into the brain (note that extra-corporeal ultrasound is challenging to repeatedly induce the BBB opening due to the need of tuning ultrasound arrays for energy-focusing and the complex operation of MRI to guide this tuning).

The ultrasonic transducer 2000, made of the composite material 100 discussed herein, such as the piezoelectric glycine-PCL mat described, further comprises a first metal electrode 2010 adjacent the composite material 100 and a second metal electrode 2020 also adjacent the composite material 100 and opposite the composite material from the first metal electrode 2010. The metal electrodes 2010, 2020 may be formed from molybdenum (Mo). The electrode 2000 then further includes an encapsulation layer 2030 covering the first metal electrode 2010, the second metal electrode 2020, and the composite material 100. The encapsulating layer 2030 typically comprises a biodegradable polymer.

The electrodes 2010, 2020, corresponding wires 2040, 2050 coupled to the electrodes, and encapsulating layers 2030, which may be formed from polylactic acid (PLA) layers, can all safely degrade inside the body after a controllable time to avoid any invasive removal surgeries which are often required for conventional ultrasonic transducers (e.g., Sonocloud37) (FIG. 21). Mo and PLA are common biodegradable materials which are used extensively in many Food and Drug Administration (FDA)-approved implants, thus affirming the safety of this device.

During use, the ultrasonic transducer 2000 described may be provided with the first metal electrode 2010 and the second metal electrode 2020 electrically coupled to an ultrasonic generator by way of the corresponding wires 2040, 2050.

The ultrasonic transducer 2000 described may then be used to deliver a therapeutic through a blood-brain barrier in a subject in need thereof. During use, the ultrasonic transducer 2000 is applied to a defect, such as a craniotomy defect, of the subject. The method then proceeds transmit an ultrasonic wave signal through the wire 2040, 2050 from the ultrasonic generator (not shown). The ultrasonic generator is then used to deliver a pulsed acoustic pressure to the craniotomy defect by way of the transducer 2000. In some embodiments, the ultrasonic wave signal is driven at about 1 MHz to about 5 MHz.

During treatment, the method may further include administering to the subject a therapeutic by way of an IV after transmitting the ultrasonic wave signal.

The perception of ultrasonic transducers for BBB disruption is schematically illustrated in FIG. 22. Microbubbles 2200 were injected intravenously into the tail vein of the animals so that the microbubbles amplify local cavitation of ultrasound pulses to transiently disrupt tight junctions between endothelial cells in the microvasculature of the brain and increase the permeability of BBB. The experimental flow and the injection timelines are presented in FIG. 23.

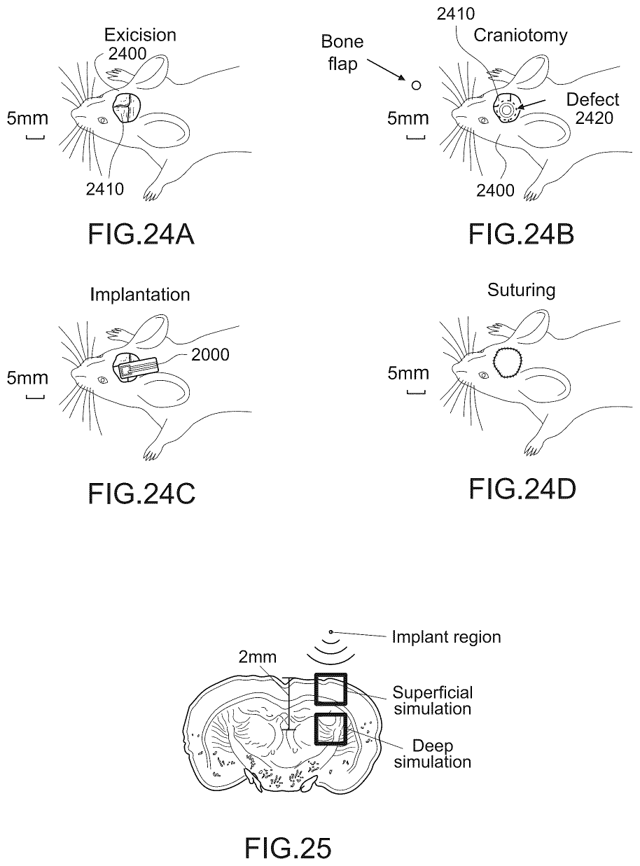

FIG. 24A-D illustrate a surgical procedure for implanting the transducer 2000 described. Excision of the epidermis 2400 from the skull 2410 followed by craniotomy were performed (FIGS. 24A and 24B), resulting in a defect 2420 in the skull 2410. A dissolvable surgical glue secured the device 2000 to the parietal bone area, as shown in FIG. 24C. The surgical site was sutured in a standard process that enabled the biodegradable Mo wires to emerge from the skin for electrical connection to external powering devices (FIG. 24D). These wires (100 μm thick) have dimensions comparable to those of the surgical threads and therefore pose little additional risk. Mice were freely moving with a stable device implant performing natural movements. The Mo wires were connected to a system (e.g., ultrasonic generator, not shown) including a function generator and a power amplifier that generates burst signals. The transducer 2000 operates at 1 MHz to generate an acoustic pressure of 0.3 MPa (rarefaction pressure value) in a series of 2 shots lasting 30 seconds, with a 30-second break between each shot. After the sonication process, dextran (10 kDa, FITC, Lysine Fixable; Thermo Fisher) as a drug model was retro-orbitally injected into the mice. The efficacy of glycine-PCL transducer to open BBB was compared with those of a bare PCL (the negative control sample) and piezoelectric PLLA (the positive control sample) transducers. All experiments were performed with the same concentration of microbubbles and dextran, and with the same therapeutic regimen.

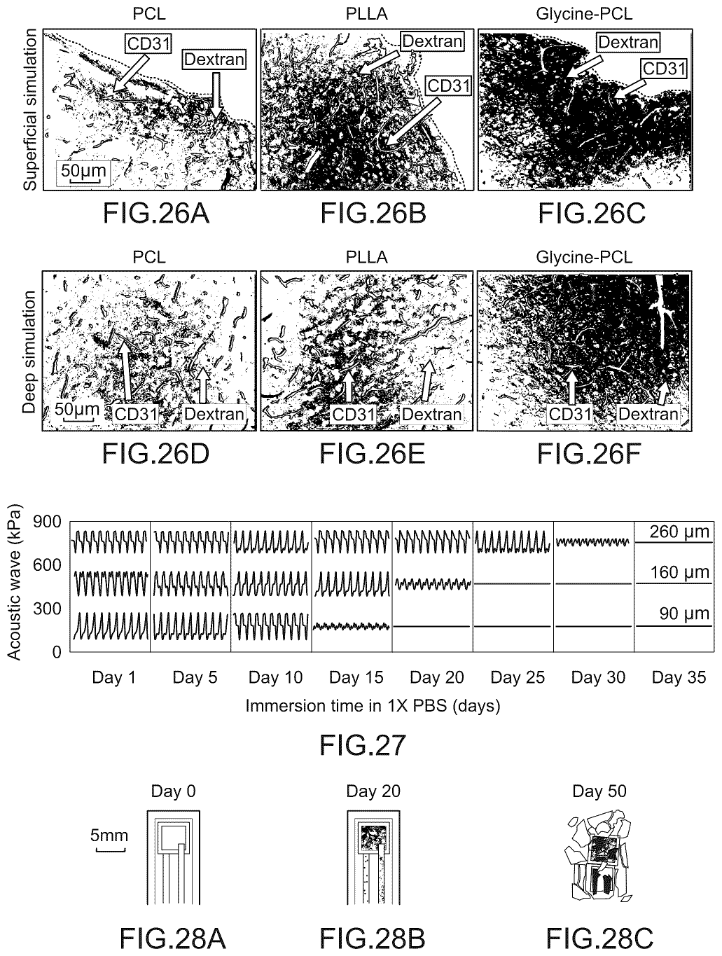

The brains were processed for immunofluorescence analysis to detect the leakage of dextran which penetrated the disrupted BBB. Two coronal brain regions, superficial and deep, which are 2 mm apart were imaged (FIG. 25). Microvessels (CD31) are distinguished by the presence of uniform red fluorescence (calcein AM), shown in the figures as outlined vessels, where dextran is detected by a bright green fluorescence (BOBO™-3 Iodide), shown in the figures as darker regions.

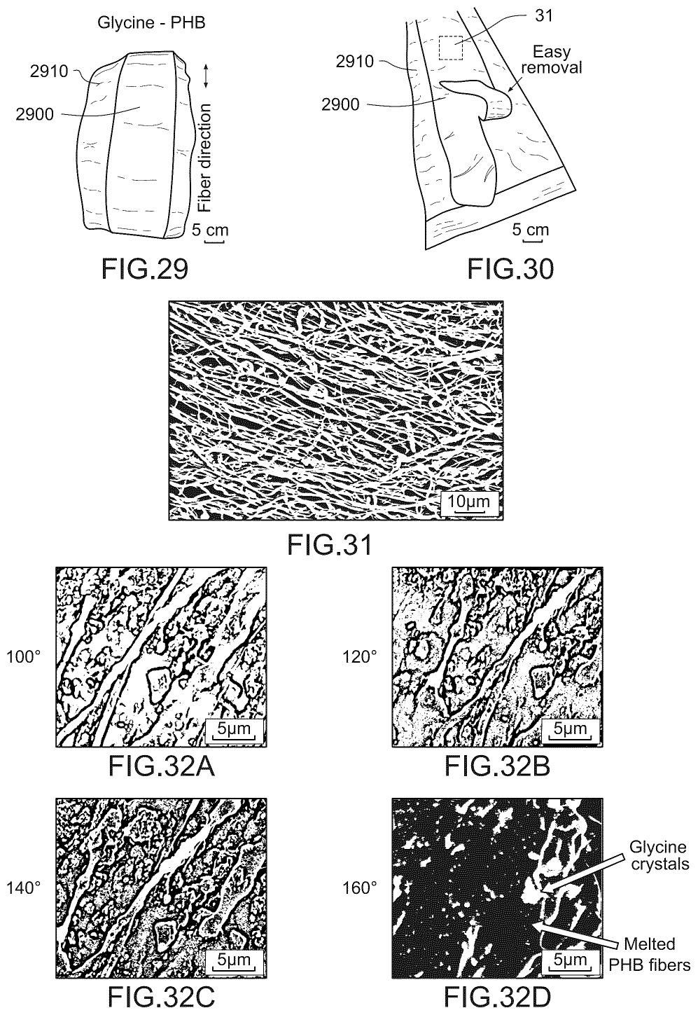

As seen in FIG. 26C, a noticeable amount of dextran (darker regions) can be seen around various microvessels in the brain of the mouse sonicated by glycine-PCL transducer. In contrast, no similar signal was observed from the same coronal sections of the control mouse, sonicated by a PCL-based transducer (FIG. 26A). Additionally, as seen in FIG. 26B, the leaked dextran across the BBB is visible in the positive control group using the piezoelectric PLLA-based transducer but much lower than glycine-PCL group with the same input/surgical conditions.

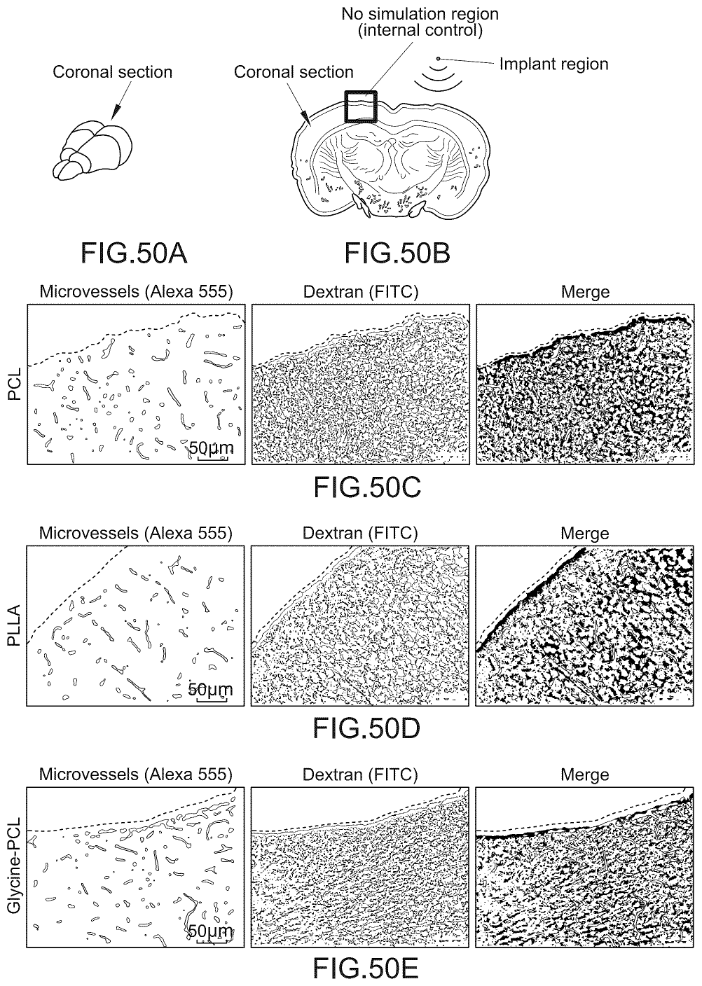

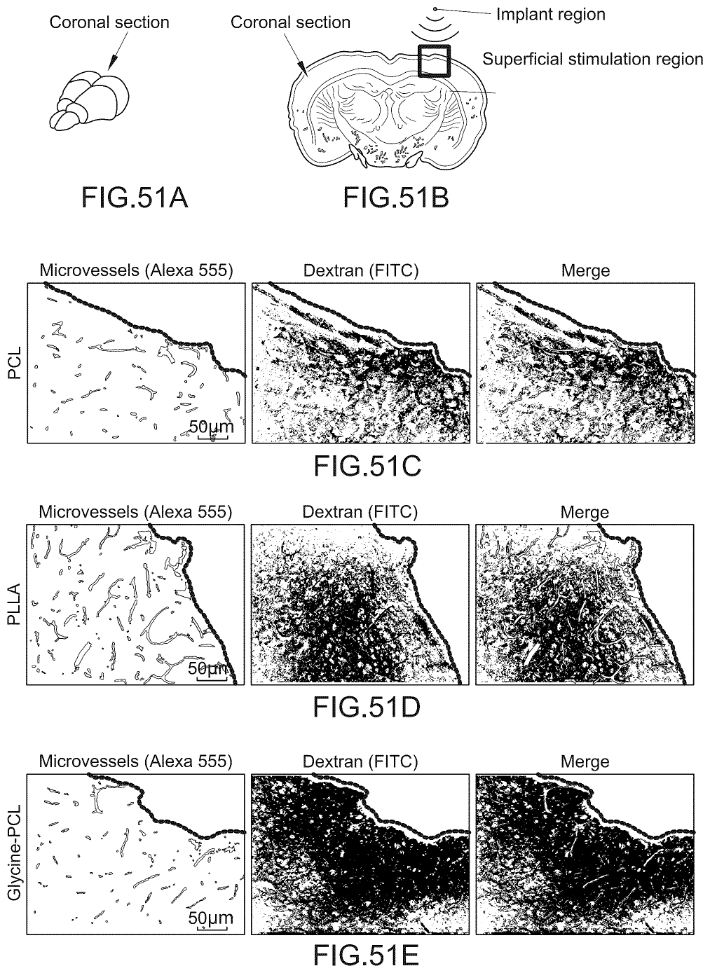

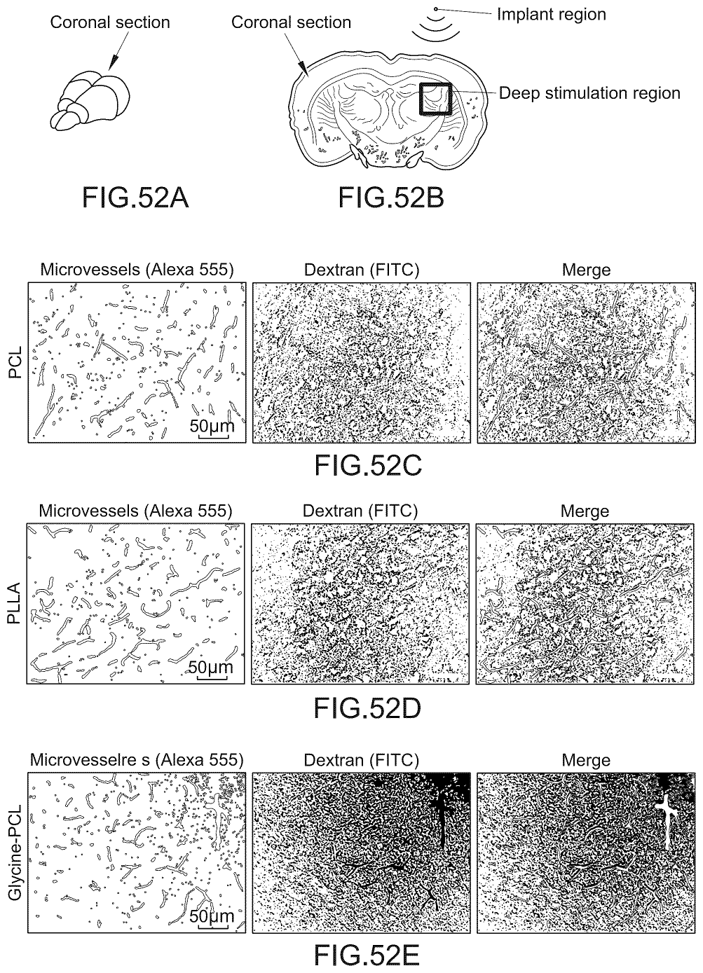

Furthermore, in order to examine the penetration depth of the generated ultrasound into the brain tissue, a 2 mm region below the brain surface is imaged. As seen in FIG. 26F, a remarkable level of dextran (darker sections of the image) was found around the microvessels in the brain of the mouse that received the treatments by glycine-PCL transducer. On the contrary, no dextran signal was detected from the same coronal sections of the PCL and the piezoelectric PLLA (state-of-the-art biodegradable ultrasonic implant for BBB opening) samples (FIGS. 26D and 26E). The notable level of BBB disruption by glycine-PCL-based transducer in the mouse model suggests the significant potential of glycine-PCL piezoelectric film for this blood-brain drug delivery application. Additional representative images of the brain sections in the experimental group, the negative and positive control groups as well as other internal control groups, are provided in FIG. 50A-52E.

Finally, a functionality experiment was conducted to determine the operational lifetime of a transducer 2000 encapsulated with 90-260 μm thick layers of PLA 2030 and demonstrated the device has a lifetime of 10-25 days in PBS at 37° C. depending on the encapsulation thickness (FIG. 27). Thus, the encapsulating PLA layers 2030 allow for control of the device degradation at well-defined rates. All the tested devices functioned well in their predefined lifetime and eventually self-degraded as illustrated in the accelerated degradation condition of FIGS. 28A-C.

Accordingly, as further illustrated in FIG. 53, when making and using the ultrasonic transducer 2000 described herein, a user would first receive the biodegradable composite material 100 described herein and then apply a first electrode 2010 to a first side of the composite material and a second electrode 2020 to a second side of the composite material 100 (5360). One or more wires 2040, 2050 may be left extending from the corresponding electrodes 2010, 2020. The user may then encapsulate (5370) the composite material 100 and the first and second electrodes 2010, 2020 with a biodegradable encapsulating layer 2030. As noted above, the encapsulating layer 2030 may be formed from a biodegradable polymer.

In doing so, a user may, in some embodiments, first determine an idealized functional lifetime (5380) of the biodegradable piezoelectric composite material 100. Such an idealized functional lifetime may depend on a use case for the biodegradable piezoelectric composite material 100. Accordingly, the functional lifetime may be based on a projected treatment process for a patient or subject (5390). Once an idealized functional lifetime is selected, the user may select a thickness (5400) for the encapsulating layer based on the idealized functional lifetime.

As noted above, following the encapsulation of the transducer 2000 in an encapsulating layer 2030, the ultrasonic transducer may be implanted (5400) adjacent a craniotomy defect 2410 of a subject as part of a method of therapy. Such a method may then proceed with transmitting (5410) an ultrasonic wave signal through one or more wire 2040, 2050 in electrical communication with the first or second electrode 2010, 2020. It is understood that although the method describes the actuation by way of an ultrasonic wave signal transmitted through a wire, other forms of actuation are contemplated as well.

In some embodiments, after transmitting the ultrasonic wave signal, the method may proceed with administering to the subject a therapeutic intravenously (5420).

Further, in some embodiments, prior to administering the therapeutic (at 5420), the subject may be injected with microbubbles (5415), which may increase the permeability of the BBB, as discussed above.

Disclosed is a unique biodegradable, soft and highly piezoelectric nanomaterial platform, made of glycine crystals 130 embedded inside a biodegradable matrix of PCL 120. While glycine crystals 130 are known to be safe and possess an attractive ultrahigh piezoelectric effect, there has not been any success to create the crystals in a flexible, easy-to-handle and stable form for useful device applications. Here, electrospinning processes were used to fabricate a soft, biodegradable, and stable glycine-embedded PCL nanofibers 140 and employ the nanofibers to create an exceptional biodegradable and powerful implantable ultrasound transducer 2000 for a significant application of blood-brain drug delivery. It was also demonstrated that the glycine-PCL nanofiber device 2000 exhibited an excellent actuation and ultrasonic performance, superior to those of the state-of-the-art biodegradable PLLA piezoelectric device that has been recently reported. Using only medical safe materials, the glycine-PCL nanofiber ultrasound device was created that is safe for cell culture and animal implantation. After a controllable and pre-defined lifetime, the glycine-PCL nanofiber device 2000 can self-degrade which is significant for implant application to avoid risky and invasive removal surgeries. Especially, the powerful piezoelectric performance of the nanofibers allowed the glycine-PCL ultrasonic devices, superficially implanted on the brain, to trigger the BBB opening even in deep brain regions. This ability could be important to the application of deep brain stimulation/modulation or the treatment of cancerous/diseased tissues deep inside the brain. Besides medical applications, the excellent piezoelectricity of this flexible, easy-to-use, and safe glycine platform could also enable the creation of “green” sensors, actuators, and transducers to replace the common lead-based or non-degradable piezoelectric devices which are used extensively in industry, and harmful to human and the environment. Overall, the disclosed glycine-PCL nanofiber platform offers a powerful and safe amino acid based piezoelectric nanomaterial which could bring about significant medical implant applications such as soft biodegradable sensors, actuators, or transducers.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

What is claimed is:

1. A composite material comprising:
a biodegradable polymer; and
a plurality of glycine crystals embedded in the biodegradable polymer,
wherein the biodegradable polymer comprises a plurality of biodegradable fibers substantially aligned with each other.

2. The composite material of claim 1, wherein each biodegradable fiber has a diameter of about 1 μm to about 5 μm.

3. The composite material of claim 1, wherein the biodegradable polymer comprises one or more of poly (L-lactic acid) (PLLA), poly(D,L-lactide-co-glycolide) (PLGA), polycaprolactone (PCL), polyglycolic acid (PGA), polyhydroxybutyrate, silk, polyvinyl alcohol, chitosan, or combinations thereof.

4. The composite material of claim 1, wherein each glycine crystal has a diameter of about 1 nm to about 5 μm.

5. The composite material of claim 1, wherein the plurality of glycine crystals are elongated and substantially aligned with each other.

6. The composite material of claim 1, wherein the material comprises a weight ratio of about 0.25:1 to about 2:1 of the plurality of glycine crystals to the biodegradable polymer.

7. The composite material of claim 1, wherein the material has an elastic modulus of about 10 MPa to about 2,000 MPa.

8. The composite material of claim 1, wherein the material has a piezoelectric output of greater than 600 kPa as measured by generated acoustic pressure.

9. The composite material of claim 2, wherein the plurality of biodegradable fibers are formed by electrospinning a mixture comprising a biodegradable polymer, a plurality of glycine crystals, and at least one solvent onto a collector drum having a speed of about 100 RPM to about 4,000 RPM at a voltage of about 10 kV to about 25 kV and at a flow rate of about 2 ml/h, at a humidity of about 30% to about 50%, or a combination thereof.

10. An ultrasonic transducer comprising the composite material of claim 1 and further comprising:
a first metal electrode adjacent the composite material;
a second metal electrode adjacent the composite material and opposite the composite material from the first metal electrode; and
an encapsulation layer covering the first metal electrode, the second metal electrode, and the composite material, wherein the encapsulation layer comprises a biodegradable polymer.

11. The ultrasonic transducer of claim 10, wherein the first metal electrode and the second metal electrode are electrically coupled to an ultrasonic generator through the one or more wire.

12. A method of delivering a therapeutic through a blood-brain barrier in a subject in need thereof, the method comprising:
applying the ultrasonic transducer of claim 11 to a craniotomy defect of the subject;
transmitting an ultrasonic wave signal through the wire; and
delivering a pulsed acoustic pressure to the craniotomy defect.

13. The method of claim 12, further comprising administering to the subject a therapeutic intravenously after transmitting the ultrasonic wave signal.

14. A method of making a biodegradable piezoelectric composite material, the method comprising:
mixing a solution comprising a biodegradable polymer, glycine crystal, and at least one solvent;
electrospinning the solution;
receiving the electrospun solution onto a collector drum having a speed of about 100 RPM to about 4,000 RPM, such that resulting biodegradable polymer fibers are substantially aligned with each other.

15. The method of claim 14, further comprising:
growing or retrieving glycine crystals, wherein the glycine crystals are needle-shaped;
grinding or otherwise crushing the glycine crystals to create glycine particles; and
incorporating the glycine particles into the solvent as the glycine crystal component.

16. A method for making an ultrasonic transducer comprising:
applying a first electrode to a first side of the biodegradable piezoelectric composite material of claim 1;
applying a second electrode to a second side of the biodegradable piezoelectric composite material opposite the first side; and
encapsulating the composite material and the first and second electrodes with a biodegradable encapsulating layer.

17. The method of claim 16 further comprising:
determining an idealized functional lifetime of the biodegradable piezoelectric composite material; and
selecting a thickness for the encapsulating layer based on the idealized functional lifetime,
wherein the idealized functional lifetime depends on a use case for the biodegradable piezoelectric composite material.

18. A method of therapy comprising:
implanting the ultrasonic transducer of claim 17 adjacent a craniotomy defect of a subject; and
transmitting an ultrasonic wave signal through a wire in electrical communication with the first or second electrode.

19. The method of claim 18 further comprising administering to the subject a therapeutic intravenously after transmitting the ultrasonic wave signal.

20. A method of therapy comprising:
implanting the ultrasonic transducer of claim 17 adjacent a craniotomy defect of a subject;
transmitting an ultrasonic wave signal to the first or second electrode;
injecting microbubbles into the subject; and
administering to the subject a therapeutic intravenously.
